(19) United States
(12) Patent Application Publication
(10) Pub. No.: US 2008/0267876 A1
(43) Pub. Date: Oct. 30, 2008

(54) Nanoparticles for Targeted Delivery of Active Agent

(75) Inventors: Shimon Benita, Mevasseret Zion (IL); Nir Debotton, Tel Aviv (IL); Danny Goldstein, Jerusalem (IL)

Correspondence Address: BROOKS KUSHMAN P.C., 1000 TOWN CENTER, TWENTY-SECOND FLOOR, SOUTHFIELD, MI, 48075, US

(73) Assignee: YISSUM RESEARCH DEVELOPMENT COMPANY, Jerusalem (IL)

(21) Appl. No.: 12/067,510

(22) PCT Filed: Sep. 20, 2006
(86) PCT No.: PCT/IL2006/001098

§ 371 Date: Jul. 17, 2008

Related U.S. Application Data
(60) Provisional application No. 60/718,333, filed on Sep. 20, 2005.

(51) Int. Cl. A61K 49/00; A61K 47/06; A61K 47/22; A61K 9/14; A61K 47/30; A61K 47/26

(52) U.S. Cl. 424/9.1; 514/772; 514/788; 424/489; 514/772.3; 514/773

(57) ABSTRACT

The present invention concerns a delivery system comprising a polymer-based nanoparticle; and a linker comprising a first portion non-covalently anchored to said nanoparticle, wherein at least part of said first portion comprises a hydrophobic/lipophilic segment embedded in said nanoparticle; and a second portion comprising a maleimide compound exposed at the outer surface of said nanoparticle. In accordance with one embodiment, the delivery system comprises one or more targeting agents, each covalently bound to said maleimide compound. In accordance with yet another embodiment, the delivery system comprises a drug. A specific example for a linker in accordance with the invention is octadecyl-4-(maleimideomethyl)cyclohexane-carboxylic amide (OMCCA).

### FIELD OF THE INVENTION

The present invention relates to polymer-based nanoparticles for use as delivery vehicles.

### LIST OF PRIOR ART

The following is a list of prior art which is considered to be pertinent for describing the state of the art in the field of the invention.
Takeshi Matsuya et al. Anal. Chem. 75:6124-6132 (2003);
Terro Soukka et al. Clinical Chemistry 47(7):1269-1278 (2001)
Terro Soukka et al. Anal. Chem. 73:2254-2260 (2001);
Arai K. et al. Drug Des. Deliv. 2(2):109-120 (1987);
Harma H. et al. Luminescence 15(6):351-355 (2000);
Olivier J C. et al. Pharm. Res. 19(8):1137-1143 (2002);
Olivier J C. NeuroRx. 2(1):108-119 (2005);
Lu Z R. et al. Nature Biotechnology 17:1101-1104 (1999);
Gref R. et al. Biomaterials 24(24):4529-4537 (2003);
Nobs L. et al. Eur. J. Pharm. Biopharm. 58(3):483-490 (2004);
Ezpeleta I. et al. Int. J. Pharm. 191(1):25-32 (1999);
Lundberg B B, et al. J Pharm Pharmacol. 51(10):1099-105 (1999);
US2005/042298;
WO1987/07150;
WO2003/088950;
U.S. Pat. No. 6,221,397;
WO2005/077422.

### BACKGROUND OF THE INVENTION

The ability to target active substances such as drugs and genes to tissues has been one of the most sought after goals in clinical therapeutics. One approach, referred to by the term “active targeting” concerns the attachment of specific ligands to the surface of colloidal for targeting to specific cells. As a result, the ligands selectively bind to surface epitopes or receptors on target sites, [Moghimi S M, et al. Pharmacol Rev. 53(2):283-318 (2001)].

Another approach emerged with the approval of monoclonal antibodies (MAb) for therapeutic applications especially in cancer [Allen T M. Nat Rev Cancer. 2(10):750-63 (2002)]. The use of MAb for the treatment of cancer was suggested as a means of targeting cancer cells while sparing normal cells. MAbs are being coupled with colloidal carriers such as liposomes (to form immunoliposomes), emulsions (to form immunoemulsions) and nanoparticles (to form immunonanoparticles). These immunoconjugates thus ensure the specific recognition of the antigen site by the antibody and the release of different cytotoxic agents by the colloidal delivery system close to the inaccessible pathological target tissues, over-expressing tumor antigen.

Immunoliposomes have already been described [Park J W, et al. J Cont Rel. 74(1-3):95-113 (2001): Park J W et al. Clin Cancer Res. 8(4):1172-81 (2002); Nam S M, et al. Oncol Res. 11(1):9-16 (1999)]. Further, it has been shown that immunoliposomes bearing polyethyleneglycol (PEG)-coupled Fab′ fragments elicited prolonged circulation time and high extravasations into targeted solid tumors in vivo [Maruyama K, et al. FEBS Lett. 413(1):177-80 (1997)]. However, these were found to be physicochemical instable. In addition, most of these liposomal carriers were unable to incorporate significant doses of lipophilic/hydrophobic active ingredients, limiting their potential clinical efficacy.

Immunoemulsions have also been described. For example, Lundberg B B et al. describes the conjugation of an anti-B-cell lymphoma monoclonal antibody (LL2) to the surface of lipid-emulsion globules by use of a poly(ethylene glycol)-based heterobifunctional coupling agent and the use of same as drug carriers [Lundberg B B, et al. J Pharm Pharmacol. 51(10):1099-105 (1999)]. Yet, lipid emulsions as such can incorporate only highly lipophilic drugs which exhibit marked poor aqueous solubility. The difficulty in retaining within the oil droplets potent moderately lipophilic cancer chemotherapy agents upon infinite dilution, limits the therapeutic applications of these dosage forms. For example, paclitaxel was found to be released rapidly form the lipid emulsion following intravenous injection [Lundberg B B. J Pharm Pharmacol. 49(1):16-20 (1997)].

A further study making use of oil emulsions involves the formation of positive oil in water emulsions; the emulsion comprising a compound presenting free NH₂ groups, at its natural state, at the oil-water interface, and an antibody, wherein the compound is linked to the antibody by a heterobifunctional linker, linking the NH₂ groups to SH groups on the antibody hinge region [Benita S. et al. International Patent Application Publication No. WO2005/077422]

Over the past few decades, there has been considerable interest in developing biodegradable and biocompatible nanoparticles (NPs) as effective drug delivery systems. Conventional NPs undergo rapid clearance following intravenous (iv) administration by the reticuloendothelial system (RES). Hydrophilic linear polyethylene glycol (PEG) molecules ranging in MW from 2000 Da to 5000 Da anchored on the particle surface and oriented towards the aqueous phase confer steric stabilization prevent opsonization and uptake of the NPs by the RES. These stealth NPs exhibited prolonged plasma circulating time [Avgoustakis K, et al. Int J Pharm. 259(1-2):115-27 (2003); Li Y, et al. J Control Release. 71(2):203-11 (2001); Matsumoto J, et al. Int J Pharm. 185(1):93-101 (1999); Stolnik S, et al. Pharm Res. 11(12):1800-8 (1994)].

NPs can entrap various hydrophilic and moderately lipophilic drugs such as vaccines, peptides, proteins, oligonucleotides and anti-tumor agents [Soppimath K S, J Control Release. 70(1-2):1-20 (2001); Brigger I, et al. Adv Drug Deliv Rev. 54(5):631-51 (2002)]. The encapsulation of anti-tumor agents in NPs has been widely investigated since NPs are suitable means for improving the therapeutic index of potent drugs while greatly reducing their side effects. Among the promising anti-tumor agents incorporated in NPs, doxorubicin [Soma C E, et al. J Control Release. 68(2):283-9 (2000)] and paclitaxel NPs [Xu Z et al. Int J Pharm. 288(2):361-8 (2005); Dong Y, Feng S S. Biomaterials. 25 (14):2843-9 (2004)] are exhibiting encouraging results.

Despite great clinical potential, the approach of targeting NPs to organs via MAb (immunonanoparticles) has not been fully exploited. The ability to selectively target anticancer drug loaded NPs via specific ligands against antigens over-expressed in malignant cells could improve the therapeutic efficacy of the immunonanoparticles (immunoNPs) preparations as well as reduce adverse side effects associated with chemotherapy.

There are few studies dealing with the covalent coupling of MAb to biodegradable NPs, and even fewer dealing with in vitro and in vivo experimentations [Nobs L, et al. J Pharm Sci. 93(8):1980-92 (2004)]. In one of these studies anti-transferrin receptor MAb was conjugated to PEGylated poly(lactic acid) NPs [Olivier J C, et al. Pharm Res. 19(8):1137-43 (2002)]. Other studies demonstrate the conjugation of MAb to poly(lactic acid) NPs via biotin-avidin interactions [Nobs L, et al. Int J Pharm. 250(2):327-37 (2003); Nobs L, et al. Eur J Pharm Biopharm. 58(3):483-90 (2004)].

### SUMMARY OF THE INVENTION

The present invention is based on the development of a simple approach for associating targeting agent, such as antibodies, to polymer-based nanoparticles (preferably those comprising a therapeutically active agent), which does not require a priori chemical binding of the targeting agent to the particle-forming polymer. This was achieved by the use of a bi-functional linker having a lipophilic portion which non-covalently anchors to the particle's polymeric matrix and a second portion comprising a maleimide compound to which it is possible in a subsequent step to bind the targeting agent. This novel approach eliminates the need to tailor for each different targeting agent a different nanoparticle composition, and enables to form a “universal” nanoparticle-linker (with an active agent such as a cytotoxic agent), which can be used to prepare different targeted systems, simply by binding to the linker different targeting agents according to needs.

Thus, according to a first of its aspects, the present invention provides a delivery system comprising:

(i) a polymer-based nanoparticle;

(ii) a linker comprising a first portion non-covalently anchored to said nanoparticle, wherein at least part of said first portion comprises a hydrophobic segment embedded in said nanoparticle; and a second portion comprising a maleimide compound exposed at the outer surface of said nanoparticle.

The nanoparticle preferably comprises an active agent carried by the particle, such as a drug, a contrasting agent and combinations of same, embedded, impregnated, or encapsulated in said particle, or adsorbed at the surface of the particle.

The above nanoparticle-linker can be used in subsequent production of the final targeted product, as the linker is suitable for covalent binding with a targeting agent.

According to one preferred embodiment, the nanoparticle comprises one or more targeting agents each covalently bound to said maleimide compound.

The invention also provides a composition comprising the delivery system of the invention. In accordance with one embodiment, the composition comprises a pharmaceutically acceptable carrier. In accordance with some other embodiments, the composition comprises an active agent carried by said nanoparticle.

The invention also provides a method for treating or preventing a disease or disorder, the method comprises providing a subject in need, an amount of the delivery system of the invention, the amount being effective to treat or prevent said disease or disorder.

Yet further, the invention provides a method of imaging in a subject's body a target cell or target tissue, the method comprising:

(a) providing said subject with the delivery system of the invention and carrying a contrasting agent wherein the nanoparticles are associated with one or more targeting agents effective to target said delivery system to said target cell or target tissue;

(b) imaging said contrasting agent in said body.

### BRIEF DESCRIPTION OF THE FIGURES

In order to understand the invention and to see how it may be carried out in practice, a preferred embodiment will now be described, by way of non-limiting examples only, with reference to the accompanying Figures, in which:

FIGS. 1A-1C are schematic illustrations of a delivery particle according to the invention, in which a linker (OMCCA) has a first portion anchored in the particle, and a second portion (maleimide) exposed at the surface of the particle and associated to an antibody (Y) (FIG. 1A); the delivery particle may further comprise portions of the polymer modified with polyethylene glycol (FIG. 1B), and may also carry a drug embedded in the polymeric matrix (FIG. 1C).

FIG. 2 is a three dimensional bar graph showing zeta potential measurements for non-conjugated particles (blank), trastuzumab-conjugated particles (immunoNPs), trastuzumab-conjugated and drug loaded particles (immuno DCTX NPs).

FIGS. 3A-3B shows transmission electron microscopy images of antibody-conjugated nanoparticles according to the invention, using 12 nm gold labeled goat anti-human IgG, at two scales, 200 nm (FIG. 3A) and 100 nm (FIG. 3B).

FIGS. 4A-4C are FITC images of Trastuzumab binding to SK-BR-3 cells visualized by FITC-conjugated anti-human IgG, after incubation of particles without trastuzumab (FIG. 4A); after incubation with immuno-particles, i.e. conjugated to trastuzumab (FIG. 4B); or after incubation with Traut modified trastuzumab-conjugated nanoparticles (FIG. 4C).

FIG. 5 shows FACS analysis for LNCaP cells incubated first with different trastuzumab amounts and followed by FITC-conjugated anti-human IgG: 1 ug, 10 ug, and 50 ug, and control.

FIGS. 6A-6B are confocal microscopy photographs of SK-BR-3 cells incubated with trastuzumab-conjugated nanoparticles with a PLA/OMCCA ratio of 50:6 mg/mg (FIG. 6A); or with trastuzumab-conjugated nanoparticles with a PLA/OMCCA ratio of 50:10 mg/mg (FIG. 6B).

FIGS. 7A-7D show images of the binding of paclitaxel-palmitate loaded trastuzumab NPs to PC3.38 from two batches obtained by bright field microscopy (FIGS. 7A-7B, first and second batch, respectively) and by fluorescence microscopy (FIGS. 7C-7D, first and second batch, respectively).

FIGS. 8A-8B show images of cellular uptake by PC-3.38 cells of coumarin-6 labeled NPs (FIG. 8A) and coumarin-6 labeled trastuzumab immunoNPs (FIG. 5B) as determined by Confocal laser scanning microscopy (CLSM).

FIGS. 9A-9D show images of cellular uptake by CAPAN-1 cells of coumarin-6 labeled NPs (FIG. 9A), AMB8LK immunoNPs (FIG. 9B) trastuzumab immunoNPs (FIG. 9C) and immunoNPs conjugated to trastuzumab and to AMB8LK (FIG. 9D) as determined by fluorescence microscopy.

FIGS. 10A-10D show images of cellular uptake by PC-3.38 cells of coumarin-6 labeled NPs (FIG. 10A), trastuzumab immunoNPs (FIG. 10B) AMB8LK immunoNPs (FIG. 10C) and immunoNPs conjugated to trastuzumab and to AMB8LK (FIG. 10D) as determined by fluorescence microscopy.

FIG. 11 is a bar graph showing cellular uptake of pcpl by PC-3.38 cells when the cells were incubated with [³H]-pcpl solution (pcpl solution); [³H]-pcpl loaded NPs (pcpl NPs) and [³H]-pcpl loaded NPs conjugated to trastuzumab (pcpl immunoNPs). Values are mean ±SD, N=5.

FIGS. 12A-12F are graphs showing pcpl concentration, either in the form of a solution (cppl solution), loaded onto NPs (pcpl NPs) or loaded on NPs conjugated to trastuzumab (pcpl immunoNPs) in different tissues: in blood, following intravenous (i.v.) injection in the indicated tissue normalized to gram tissue, 5 minutes post i.v. injection (FIG. 12A); 1 hour post i.v. injection (FIG. 12B); 2 hour post i.v. injection (FIG. 12C); 6 hour post i.v. injection (FIG. 12D); 24 hour post i.v. injection (FIG. 12E); and 48 hour post i.v. injection (FIG. 12F). Values are mean-SD, N=4.

### DETAILED DESCRIPTION OF THE INVENTION

The present invention is aimed to provide improvement of drug delivery therapy which is based on a novel one-step conjugation process of one or more targeting agents to drug-loaded nanoparticles. In particular, the invention enables the preparation of a universal nanoparticle linker (optionally in combination with a drug) that can be subsequently bound to a targeting agent of choice, so that there is no need to design a special nanoparticle for each different targeting agent. The design nanoparticles in accordance with the invention allow a better recognition of targeted cells exhibiting two surface membrane low antigen densities.

The present invention thus provides delivery systems comprising a polymer based nanoparticle and a linker comprising a first portion non-covalently anchored to said nanoparticle, wherein at least part of said first portion comprises a hydrophobic segment embedded in said nanoparticle; and a second portion comprising a maleimide compound exposed at the outer surface of said nanoparticle.

Maleimides are a group of organic compounds with a 2,5-pyrroledione skeleton as depicted in general formula (I) hereinbelow.

Maleimides are used in a wide range of applications ranging from advanced composites in the aerospace industry to their use as reagents in synthesis. For example the aerospace industry requires materials with good thermal stability and a rigid backbone both of which are provided by bismaleimides. In some applications, various linkers such as polysiloxanes and phosphonates are conjugated to the bismaleimides to strengthen polymers made therefrom, etc.

Maleimides may also be linked to polyethylene glycol chains which are often used as flexible linking molecules to attach proteins to surfaces. The double bond readily reacts with the thiol group found on cysteine to form a stable carbon-sulfur bond. Attaching the other end of the polyethylene chain to a bead or solid support allows for easy separation of protein from other molecules in solution, provided these molecules do not also possess thiol groups.

In the context of the present invention, maleimide is conjugated to a linker to be incorporated non-covalently into a polymer based nanoparticle and the combination of the maleimide-linker with the nanoparticle provides a delivery system platform for various active agents.

The term “delivery system” which may be used herein interchangeably with the term “delivery nanoparticles” denotes physiologically acceptable, polymer-based nanoparticles which when associated with a linker, the particles have a diameter of 1 micrometer or less, preferably in the range of about 50-1000 nm, more preferably in the range of about 200-300 nm. While the nanoparticles preferably have a matrix structure formed from one or more polymers; the term nanoparticles may also refer to nanocapsules having a core-shell structure, where the shell of the particles is formed from the polymer having an internal space (e.g. oil phase) carrying an active agent, or to a combination of same. The latter formulation may be applicable, for example, for delivery of oil miscible drugs.

Further, while the nanoparticles may be formed from substances other than a polymer, it is to be understood that the particles are essentially polymer-based or at least their outer surface is polymer-based. Thus, the term “nanoparticles” in the context of the invention excludes liposomes or emulsion forms.

The terms “polymer based particles”, “polymer based nanoparticles” or “particle-forming polymer” as used herein denotes any biodegradable, and preferably biocompatible polymer capable of forming, under suitable conditions, nanoparticles which include, without being limited thereto, either nanospheres or nanocapsules. Nanospheres (defined as polymeric spherical matrices) and nanocapsules (defined as tiny oil cores surrounded by a distinct wall polymer) are just a few of the shapes that may be obtained and used with the delivery platform disclosed herein. In accordance with some preferred embodiments it is preferable that at least the outer wall of the particle comprises in its majority one or more polymers. Thus, when the particle may comprise an oil phase core, the latter will be encapsulated within a polymer-based wall. A variety of biodegradable polymers is available in the art and such polymers are applicable in the present invention. Approved biodegradable, biocompatible and safe polymers largely used in nanoparticle preparations are described by Gilding D K et al. [Gilding D K et al. Polymer 20:1459-1464 (1979)].

Non-limiting examples of particle-forming biodegradable polymers are polyesters such as, without being limited thereto, polyhydroxybutyric acids, polyhydroxyvaleric acids; polycaprolactones; polyesteramides; polycyanoacrylates; poly(amino acids); polycarbonates; polyanhydrides; and mixtures of same.

Preferably, the polymer is selected from polylactic acid (polylactide), polylactide-polyglycolide, polyglycolide, poly(lactide-co-glycolide), polyethylene glycol-co-lactide (PEG-PLA) and mixtures of any of same.

A further component within the delivery system is the linker comprising a first portion non-covalently anchored to the nanoparticle and a second portion comprising a maleimide compound exposed at the outer surface of said nanoparticle. The first portion is configured such that at least part of same comprises a hydrophobic segment embedded in the nanoparticle's surface.

The term “anchor” as used herein denotes the penetration of at least part of the first portion of the linker through the particle's outer surface so as to obtain a stable association between the linker and the particle. The anchoring may be achieved by the incorporation of a moiety (herein termed “the anchor moiety”) at the first portion of the linker which has similar physical characteristics as the polymer. Those versed in chemistry will know how to select an anchor moiety to be compatible with the substance from which the particle is essentially made. For example, when using a hydrophobic polymer to form a particle matrix, a preferred selection of an anchor moiety is a hydrophilic and/or lipophilic moiety. In other words the anchor moiety should preferably be compatible with the polymer and eventually with the incorporated drug.

The association between the anchor moiety and the particle is preferably by mechanical fixation (e.g. by embedment) of the anchor to the polymer matrix or polymer wall (the latter, in case of nanocapsules). The mechanical fixation is obtained upon formation of the particles, when using the polymer in combination with the linker during polymer solidification process. Once the polymer solidifies in the form of particulates, it “captures” the anchor moiety of the linker to form the resulting delivery system of the invention.

The linker in the context of the present invention is an amphipathic molecule, i.e. a molecule having a hydrophobic/lipophilic portion (providing the anchor) and a maleimide compound forming part of the hydrophilic portion. It is noted that in the following whenever the term “lipophilic” is used, it may be understood interchangeably with the term hydrophilic, as long as the hydrophobic/lipophilic moiety is compatible with the polymer forming the nanoparticle. Thus, a lipophilic portion may equally refer to a hydrophilic portion. In accordance with some embodiments, the hydrophobic/lipophilic portion comprises a hydrocarbon or a lipid comprising at least 8 carbon atoms in the hydrocarbon backbone. An exemplary range is C₈-C₃₀ carbon atoms. The lipophilic moiety may be a saturated or unsaturated hydrocarbon, linear, branched and/or cyclic.

It is noted that the linker may have one or more anchors which may be incorporated in the nanoparticle's surface. For example, a double anchor may be achieved by the use of linker comprising 1,2-Distearoyl-sn-Glycero-3-Phosphoethanolamine-N-[Maleimide(Polyethylene Glycol)2000], shown in Table 1 below, which contains two lipophilic moieties.

The linker has also a second portion to which a targeting agent (as disclosed below) binds. The binding of a targeting agent is preferably by covalent attachment, although non-covalent association may, at times, also be applicable. Covalent attachment is achieved by the inclusion in the hydrophilic portion of a chemically reactive group, in the instant invention, maleimide. Maleimide may form a stable thio-ether linkage with thiol groups of targeting agents.

According to some embodiments, the linker has the following general formula (I):

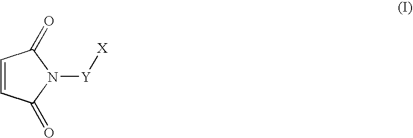

wherein

Y represents a heteroatom, a C₁-C₂₀ alkylene or alkenylene, a C₅-C₂₀ cycloalkylene or cycloalkenylene, C₆-C₂₀ alkylene-cycloalkykylene, wherein one of the carbon atoms in said alkylene or alkenylene may be replaced by a heteroatom;

X represents a carbonyl containing moiety selected from —C(O)—R₁, —C(O)—NH—R₁, —C(O)—O—C(O)—R₁, C(O)NH—R₂—R₁, or —C(O)—NH—R₂—C(O)—NH—R₁, wherein R₁ represents a hydrocarbon or a lipid comprising at least 8 carbons and R₂ represents a hydrophilic polymer.

In accordance with such embodiments, R₁ may represent a lipid; R2 a hydrophilic polymer. According to one embodiment, the lipid is selected from mono or diacylglycerol, a phospholipid, a sphingolipid, a sphingophospholipid or a fatty acid.

It is noted that R₁ should be compatible with the polymer nanoparticle matrix and should be lipophilic. In accordance with this embodiment, Y may preferably represent an alkylene-cyclohexane.

The hydrophilic polymer may be any surface modifier polymer. Polymers typically used as surface modifiers include, without being limited thereto: polyethylene glycol (PEG), polysialic acid, polylactic (also termed polylactide), polyglycolic acid (also termed polyglycolide), apolylactic-polyglycolic acid, polyvinyl alcohol, polyvinylpyrrolidone, polymethoxazoline, polyethyloxazoline, polyhydroxyethyloxazoline, polyhydroxypropyloxazoline, polyaspartamide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose. The polymers may be employed as homopolymers or as block or random copolymers.

Preferably, the hydrophilic polymer is polyethylene glycol (PEG). The PEG moiety preferably has a molecular weight from about 750 Da to about 20,000 Da. More preferably, the molecular weight is from about 750 Da to about 12,000 Da and most preferably between about 2,000 Da to about 5,000 Da.

Preferably the polyethylene glycol is monomethoxypolyethylene glycol (monomethoxy or regular peg) Thus, a preferred lipopolymer utilized in accordance with the invention is stearylamine-monomethoxypoly(ethyleneglycol) (SA-mPEG).

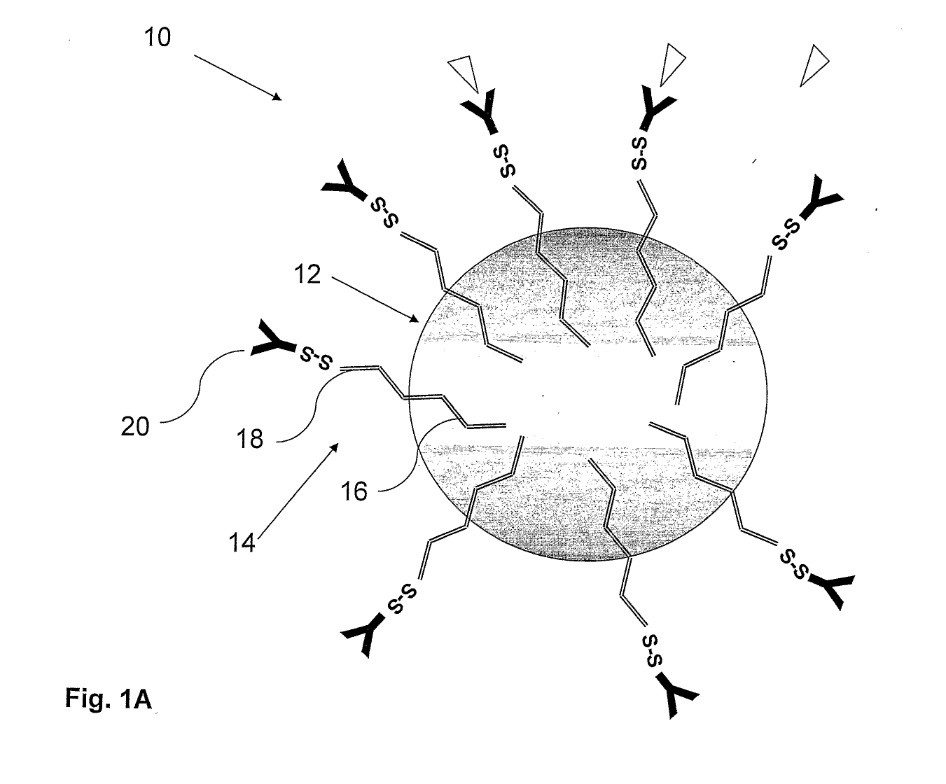

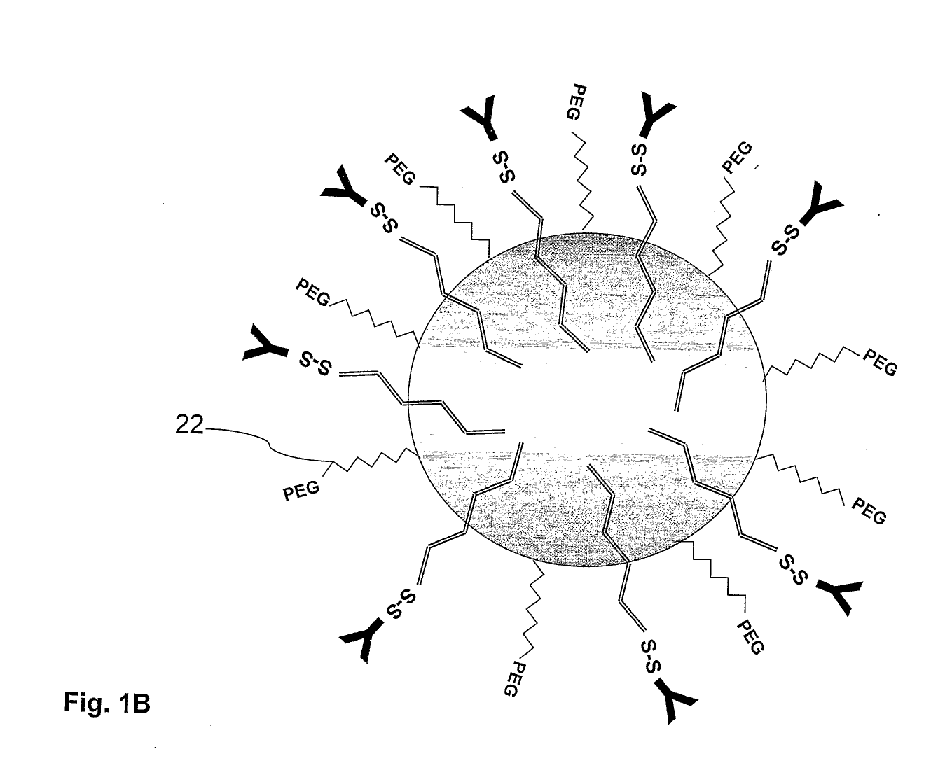

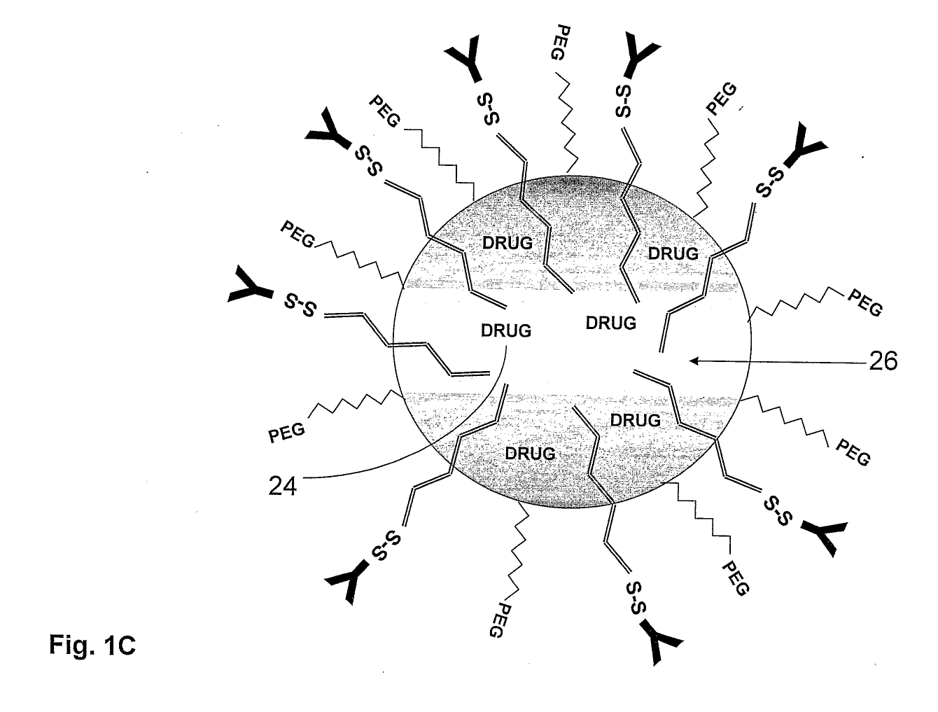

Alternatively, the hydrophilic polymer may be covalently to the polymer forming the particle, for example mPEG-polylactide, as schematically illustrated in FIG. 1B.

One particular embodiment of the invention concerns a compound of formula (I) wherein Y represents an alkylene-cycloalkykylene having the formula —CH₂—C₆H₁₀—; X represents a carbonyl containing moiety having the formula —C(O)—NH—R₁, wherein R₁ is a fatty acid.

Another particular embodiment of the invention concerns a compound of formula (I) wherein the linker is selected from Octadecyl-4-(maleimidomethyl)cyclohexane-carboxylic amide (OMCCA); N-1 stearyl-maleimide (SM); succinimidyl oleate; 1,2-Distearoyl-sn-Glycero-3-Phosphoethanolamine-N-[Maleimide(Polyethylene Glycol)2000]; and mixtures thereof (Table 1):

The chemical structures of some applicable linkers are provided in the following Table 1.

TABLE 1
Chemical names and structures of linkers

| Name | Structure |
|---|---|
| Octadecyl-4-(maleimidomethyl)cyclohexanecarboxylic amide (OMCCA) | 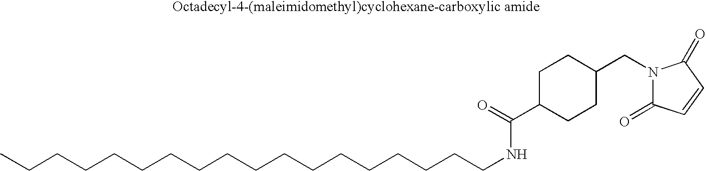 |
| Succinimidyl oleate | 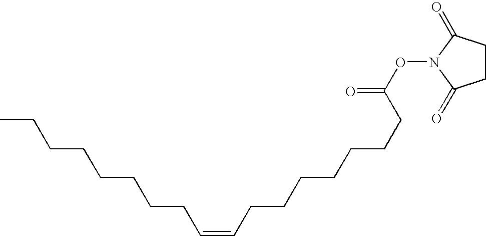 |
| Stearyl amine succinimidyl, 1,2-Distearoyl-sn-Glycero-3-Phosphoethanolamine-N-[Maleimide(PolyethyleneGlycol)2000] | 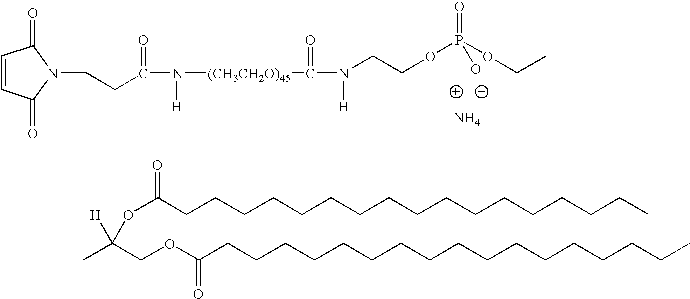 |

OMCCA, which is one preferred linker in accordance with the invention may be synthesized according to Scheme 1 below:

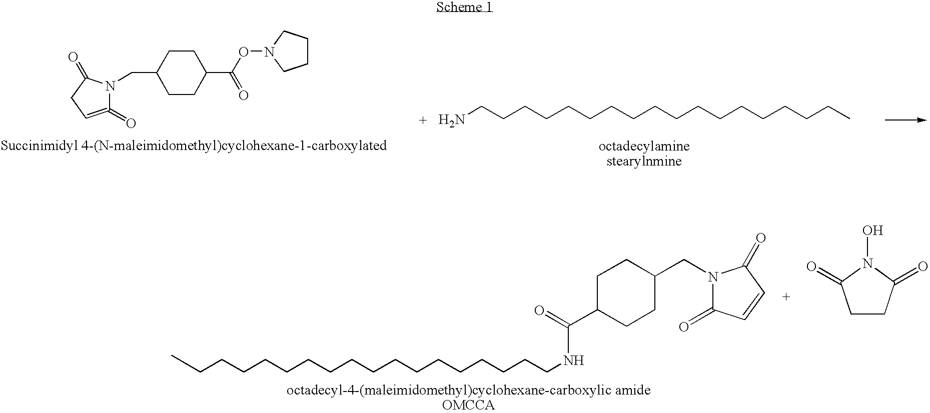

Succinimidyl oleate is commercially available from Sigma (Sigma Chemical, MO, USA; 1,2-Distearoyl-sn-glycero-3-phosphoethanolamine-N-[maleimide(polyethylene glycol)2000] is commercially available from AVANTI Polar Lipids inc, (Avanti Polar Lipids, Alabaster, Ala.).

The delivery system of the invention may be provided in the form of a targeted delivery system, i.e. a delivery system attached to a targeting agent. At times, when the targeting agent is an antibody or a binding fragment thereof, the targeted delivery system of the invention may be referred as “Immunonanoparticles”

The targeting agent may be regarded as one member of a binding couple the other member of the couple being the target on the cells, tissue to which the targeted delivery system of the invention should be selectively/preferably delivered. The term “binding couple” as used herein, signifies two substances, which are capable of specifically (affinity) binding to one another. Non-limiting examples of binding couples include biotin-avidin, antigen-antibody, receptor-ligand, oligonucleotide-complementary oligonucleotide, sugar-lectin, as known to those versed in the art.

The targeting agent may be a targeting polymer or oligomer. Non-limiting examples of polymers (and immunological functional fragments thereof) comprises amino acid-based polymers (e.g. antibodies, antigens, glycoproteins), nucleic acid-based polymers (e.g. immunostimulatory oligodeoxynucleodites (ODN), sense and antisense, interference RNA (iRNA) etc. or saccharide-based polymers, such as glycoproteins (e.g. lectins).

As noted above, also fragments of any of the above targeting may be used in accordance with the invention as long as they retain their specific binding properties to the target. When the targeting agent is an antibody (see definition below), the latter may be any one of the IgG, IgM, IgD, IgA, and IgG antibody, including polyclonal antibodies or monoclonal antibodies. Fragments of the antibodies may comprise the antigen-binding domain of an antibody, e.g. antibodies without the Fc portion, single chain antibodies, fragments consisting of essentially only the variable, antigen-binding domain of the antibody, etc.

In accordance with some embodiments, the targeting agent is a low molecular weight compound such as folic acid or thiamine. For example, thiamine may be bound to the linker anchored to the polymer based nanoparticle; and the thus formed nanoparticle, will then be specifically targeted to tissues having elevated expression of the thiamine receptor. Such target cells may include cancer cells.

In some preferred embodiments, the targeting agent is a protein associated to the particle via the linker. When referring to immunonanoparticles, the targeting agent is preferably an antibody associated with the particle via covalent binding to the linker (the linker being non-covalently attached to the particle). The other member of the binding couple is an antigen to which the antibody specifically binds. As indicated above, the targeting agent may also be an immunological fragment of an antibody.

In the context of the present invention, the term “antibody” means a substantially intact immunoglobulin derived from natural sources, from recombinant sources or by the use of synthetic means as known in the art, all resulting in an antibody which is capable of binding an antigenic determinant. The antibodies may exist in a variety of forms, including, e.g., polyclonal antibodies, monoclonal antibodies, single chain antibodies, light chain antibodies, heavy chain antibodies, bispecific antibodies or humanized antibodies; as well as immunological fragments of any of the above [Antibodies: A Laboratory Manual, Cold Spring Harbor Laboratory Press, NY; Harlow et al. (1989), Antibodies: A Laboratory Manual, Cold Spring Harbor, N. Y. Houston et al. (1988), Proc. Natl. Acad. Sci. USA 85: 5879-5883; Bird et al. (1988), Science 242: 423-426)].

As used herein, the term “immunological fragment” refers to a functional fragment of an antibody that is capable of binding an antigenic determinant. Suitable immunological fragments may be, for example, a complementarity-determining region (CDR) of an immunoglobulin light chain (“light chain”), a CDR of an immunoglobulin heavy chain (“heavy chain”), a variable region of a light chain, a variable region of a heavy chain, a light chain, a heavy chain, an Fd fragment, and immunological fragments comprising essentially whole variable regions of both light and heavy chains, such as Fv, single-chain Fv (scFv), Fab, Fab′, F(ab)₂ and F(ab′)₂.

According to a preferred embodiment of the invention, the antibody is a monoclonal antibody (MAb). The antibody may be a native protein or a genetically engineered product (i.e. recombinant antibody) or an antibody produced against a synthetic product.

Non-limiting examples of MAb which may be used in accordance with the invention are Bevacizumab, Omalizumab, Rituximab, Trastuzumab (all Genentech Inc.) AMB8LK (MAT Evry, France), Muromonab-CD3 (Johnson&Johnson), Abciximab (Centocor), Rituximab (Biogen-IDEC), Basiliximab (Novartis), Infliximab (centrocor), Cetuximab (Imclone Systems), Daclizumab (Protein Design Labs), Palivizumab (MedImmune), Alemtuzumab (Millenium/INEX), Gemtuzumab ozogamicin (Wyeth), Ibritumomab tiuxetan (Biogen-IDEC), Tositumomab-1131 (Corixa) and Adalimumab (Abbot).

More preferably the MAb is trastuzumab. Trastuzumab is a MAb with high affinity towards HER/neu tumor antigen, the latter over-expressed in malignant cells, such as in prostate cancer cells. Thus, according to one embodiment of the invention, the delivery system may be used to delivery a cytotoxic agent to cells presenting HER/neu tumor antigen.

According to some embodiments, the NP's carry two antibodies with different binding properties (e.g. different binding specificities). This structure of two different antibodies on a single nanoparticle created a “functional bispecific-like” antibody construct where the two antibodies are placed in vicinity to each other by the nanoparticle, in a relatively simple and inexpensive manner, without the need to chemically conjugate or genetically engineered a truly bi-specific single molecule

In this context, also diabodies may be used. Diabodies are a class of small bivalent and bispecific antibody fragments that can be expressed in bacteria (E. coli) and yeast (Pichia pastoris) in functional form and with high yields. Diabodies comprise a heavy (VH) chain variable domain connected to a light chain variable domain (VL) on the same polypeptide chain (VH-VL) connected by a peptide linker that is too short to allow pairing between the two domains on the same chain. This forces paring with the complementary domains of another chain and promotes the assembly of a dimeric molecule with two functional antigen binding sites. To construct bispecific diabodies the V-domains of antibody A and antibody B are fused to create the two chains VHA-VLB, VHB-VLA. Each chain is inactive in binding to antigen, but recreates the functional antigen binding sites of antibodies A and B on pairing with the other chain.

The nanoparticles of the present invention can be formed by various methods, for example: polymer interfacial deposition method, solvent evaporation, spray drying, coacervation, interfacial polymerization, and other methods well known to those ordinary skilled in the art.

Preferably the nanoparticles of the present invention are prepared by polymer interfacial deposition method as described by Fessi H et al. [Fessi H. et al. Int. J. Pharm. 1989; 55: R1-R4, The nanoparticles of the present invention may be prepared as disclosed in U.S. Pat. Nos. 5,049,322 and 5,118,528].

According to the procedure by Fessi H. et al. the particle forming polymer is dissolved in a water-miscible organic solvent: such as acetone, tetrahydrofuran (THF), acetonitrile. To this polymer containing organic phase a linker as defined above is added. The resulting organic phase is then added to an aqueous phase containing a surfactant to form dispersion, following by mixing at 900 rpm, for 1 hour, and then evaporated under reduced pressure to form nanoparticles which are then washed with a suitable buffer, such as phosphate buffered saline (PBS). The organic phase may also comprise other surfactants as well as a combination of organic solvents so as to facilitate the dissolution of an active agent to be carried by the delivery system of the invention. Similarly, the aqueous phase may contain a combination of surfactants, all of which being as described by Fessi et al.

As indicated, the delivery particle preferably carries one or more active agents. To this end, dry active agent is added to the organic phase prior to, or together with, the addition of the linker.

In order to enable formation of the nanoparticles the polymer and active agent (if incorporated) should preferably be soluble in the organic phase and insoluble in an aqueous phase, while the organic solvent and aqueous phase should be miscible.

It was found that by mere mixing the above three components, i.e. the particle forming polymer, the active agent and the linker, an amount the linker is exposed at the surface of the particle, which amount is sufficient to allow chemical binding of a targeting agent at the surface of the particles. Thus, to the forming particles (loaded with an active agent) a targeting agent is chemically associated by providing suitable conditions to allow its cross-reaction with the reactive group of the linker, exposed at the surface of the particle.

FIGS. 1A-1C are schematic illustrations of a delivery particle according to some embodiments of the invention. FIG. 1A provides a delivery particle (10) having at its outer surface (12) a linker (14) having a first portion (16) anchored in the particle through the outer surface, and a second portion (18) exposed at said surface, to which a targeting agent (20) is chemically bound. In this particular illustration, the linker is OMCCA, having a lipophilic anchored in the particle, and a maleimide moiety exposed at the surface. Maleimide may be chemically bound to the targeting agent via the formation of e.g. a sulfide bridge with a free thiol group at the targeting agent. FIG. 1B illustrates a delivery particle identical to that of FIG. 1A, however, having at its surface hydrophilic groups (22), such as PEG, to, inter alia, increase the circulation time of the particle in the body as appreciated by those versed in the art of drug delivery vehicles. FIG. 1C illustrates a delivery particle identical to that of FIG. 1B, however also indicating that a drug (24) is embedded within the internal matrix (26) of the particle.

It will be appreciated that while FIGS. 1A-1C illustrate that the first portion of the linker is fully embedded in the particle, this portion may also be partially entrapped in the particles' matrix or entrapped or encapsulated in the core core. The only prerequisite is that the anchoring is essentially stable, i.e. that the linker cannot desorb from the particle.

There is a wide variety of active agents which may be carried by the delivery particle of the invention. Carrying may be achieved by embedment of the active agent (cluster or non-clusters of the active agent) in the polymer matrix, adsorption at the surface of the particle, dispersion of the active agent in the internal space of the particle, dissolution of the active agent within the polymer forming the particle, encapsulation in the oily core of the nanoparticle etc., as known to those versed in the art.

The active agent may be a drug (therapeutic or prophylactic agent), or a diagnostic (contrasting) agent. The following is a non-limiting list of possible classes of drugs and compounds which may be loaded into the particle of the invention: analgesics, anesthetics, anti-inflammatory agents, anthelmintics, anti-arrhythmic agents, antiasthma agents, antibiotics (including penicillins), anticancer agents (including Taxol), anticoagulants, antidepressants, antidiabetic agents, antiepileptics, antihistamines, IS antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, peptides and polypeptides, parasympathomimetics, parathyroid calcitonin and biphosphonates, prostaglandins, radio-pharmaceuticals, hormones, sex hormones (including steroids), time release binders, anti-allergic agents, stimulants and anoretics, steroids, sympathomimetics, thyroid agents, vaccines, vasodilators, and xanthines

Active agents to be administered in an aerosol formulation are preferably selected from the group consisting of proteins, peptide, bronchodilators, corticosteroids, elastase inhibitors, analgesics, anti-fungals, cystic-fibrosis therapies, asthma therapies, emphysema therapies, respiratory distress syndrome therapies, chronic bronchitis therapies, chronic obstructive pulmonary disease therapies, organ-transplant rejection therapies, therapies for tuberculosis and other infections of the lung, fungal infection therapies, respiratory illness therapies associated with acquired immune deficiency syndrome, an oncology drug, an anti-emetic, an analgesic, and a cardiovascular agent.

Anti-cancer active agents are preferably selected from alkylating agents, antimetabolites, natural products, hormones and antagonists, and miscellaneous agents, such as radiosensitizers. Examples of alkylating agents include: (1) alkylating agents having the bis-(2 chloroethyl)-amine group such as, for example, chlormethine, chlorambucile, melphalan, uramustine, mannomustine, extramustinephoshate, mechlore-thaminoxide, cyclophosphamide, if osfamide, and trifosfamide; (2) alkylating agents having a substituted aziridine group such as, for example, tretamine, thiotepa, triaziquone, and mitomycine; (3) alkylating agents of the alkyl sulfonate type, such as, for example, busulfan, piposulfan, and piposulfam; (4) alkylating N-alkyl-N-nitrosourea derivatives, such as, for example, carmustine, lomustine, semustine, or; streptozotocine; and (5) alkylating agents of the mitobronitole, dacarbazine and procarbazine type.

Examples of anti-metabolites include: (1) folio acid analogs, such as, for example, methotrexate; (2) pyrimidine analogs such as, for example, fluorouracil, floxuridine, tegafur, cytarabine, idoxuridine, and flucytosine; and (3) purine derivatives such as, for example, mercaptopurine, thioguanine, azathioprine, tiamiprine, vidarabine, pentostatin, and puromycine.

Examples of natural products include: (1) vinca alkaloids, such as, for example, vinblastine and vincristine; (2) epipodophylotoxins, such as, for example, etoposide and teniposide; (3) antibiotics, such as, for example, adriamycine, daunomycine, doctinomycin, daunorubicin, doxorubicin, mithramycin, bleomycin, and mitomycin; (4) enzymes, such as, for example, L-asparaginase; (5) biological response modifers, such as, for example, alpha-interferon; (6) camptothecin; (7) taxol; and (8) retinoids, such as retinoic acid.

Examples of hormones and antagonists include: (1) adrenocorticosteroids, such as, for example, prednisone; (2) progestins, such as, for example, hydroxyprogesterone caproate, medroxyprogesterone acetate, and megestrol acetate; (3) estrogens, such as, for example, diethylstilbestrol and ethinyl estradiol; (4) anti-estrogens, such as, for example, tamoxifen; (5) androgens, such as, for example, testosterone propionate and fluoxymesterone; (6) anti-androgens, such as, for example, flutamide; and (7) gonadotropin-releasing hormone analogs, such as, for example, leuprolide. i Examples of miscellaneous agents include: (1) radiosensitizers, such as, for example, 1,2,4-benzotriazin-3-amine 1,4-dioxide (SR 4889) and 1,2,4-benzotriazine 7-amine 1,4-dioxide (WIN 59075); (2) platinum coordination complexes such as cisplatin and carboplatin; (3) anthracenediones, such as, for example, mitoxantrone; (4) substituted ureas, such as, for example, hydroxyrurea; and (5) adrenocortical suppressants, such as, for example, mitotane and aminoglutethimide.

In addition, the anticancer agent can be an immunosuppressive drug, such as, for example, cyclosporine, azathioprine, sulfasalazine, methoxsalen, and thalidomide.

Analgesic active agents, include, for example, an NSAID or a COX-2 inhibitor. Exemplary NSAIDS that can be formulated in particle of the invention include, but are not limited to, suitable nonacidic and acidic compounds. Suitable nonacidic compounds include, for example, nabumetone, tiaramide, proquazone, bufoxamac, flumizole, epirazole, tinoridine, timegadine, and dapsone. Suitable acidic compounds include, for example, carboxylic acids and enolic acids. Suitable carboxylic acid NSAIDs include, for example: (1) salicylic acids and esters thereof, such as aspirin, diflunisal, benorylate, and fosfosal; (2) acetic acids, such as phenylacetic acids, including diclofenac, alclofenac, and fenclofenac; (3) carbo- and heterocyclic acetic acids such as etodolac, indomethacin, sulindac, tolmetin, fentiazac, and tilomisole; (4) propionic acids, such as carprofen, fenbulen, flurbiprofen, ketoprofen, oxaprozin, suprofen, tiaprofenic acid, ibuprofen, naproxen, fenoprofen, indoprofen, and pirprofen; and (5) fenamic acids, such as flutenamic, mefenamic, meclofenamic, and niflumic. Suitable enolic acid NSAIDs include, for example: (1) pyrazolones such as oxyphenbutazone, phenylbutazone, apazone, and feprazone; and (2) oxicams such as piroxicam, sudoxicam, isoxicam, and tenoxicam.

Exemplary COX-2 inhibitors include, but are not limited to, celecoxib (SC-58635, CELEBREX, Pharmacia/Searle & Co.), rofecoxib (MK 966, L-74873 1, VIOXX, Merck & Co.), meloxicam (MOBIC@, co-marketed by Abbott Laboratories, Chicago, Ill., and Boehringer Ingelheim Pharmaceuticals), valdecoxib (BEXTRA@, G.D. Searle & Co.), parecoxib (G.D. Searle & Co.), etoricoxib (MK-663; Merck), SC-236 (chemical name of 4-&lsqb; 5-(4-chlorophenyl)-3-; (trifluoromethyl)-1H-pyrazol-1-yl)&rsqb; benzenesulfonamide; G.D. Searle & Co., Skokie, Ill.); NS-398 (N-(2-cyclohexyloxy-4-nitrophenyl)methane sulfonamide; Taisho Pharmaceutical Co., Ltd., Japan); SC-58125 (methyl sulfone spiro(2.4)hept-5-ene I; i Pharmacia/Searle & Co.); SC-57666 (Pharmacia/Searle & Co.); SC-558 (Pharmacia/Searle & Co.); SC-560 (Pharmacia/Searle & Co.); etodolac (Lodine, Wyeth-Ayerst Laboratories, Inc.); DFU (5,5-dimethyl-3-(3-fluorophenyl)-4-(4-i methylsulfonyl)phenyl 2(5H)-furanone); monteleukast (MK-476), L-745337 ((5 methanesulphonamide-6-(2,4-difluorothio-phenyl)-1-indanone), L-761066, L-761000, L-748780 (all Merck & Co.); DUP-697 (5-Bromo-2-(4-fluorophenyl)-3-(4 (methylsulfonyl)phenyl; DuPont Merck Pharmaceutical Co.); PGV 20229 (1-(7-tertbutyl-2,3-dihydro-3,3-dimethylbenzo(b)furan-5-yl)-4-cyclopropylbutan-1-one; Procter; & Gamble Pharmaceuticals); iguratimod (T-614; 3-formylamino-7-&rsqb; methylsulfonylamino-6-phenoxy-4H-1-benzopyran-4-one; Toyama Corp., Japan); BF 389 (Biofor, USA); CL 1004 (PD 136095), PD 136005, PD 142893, PD 138387, and PD 145065 (all Parker-Davis/Warner-Lambert Co.); flurbiprofen (ANSAID; Pharmacia & Upjohn); nabumetone (FELAFEN; SmithKline Beecham, plc); flosulide (CGP 28238; Novartis/Ciba Geigy); piroxicam (FELDANE; Pfizer3; diclofenac (VOLTAREN and CATAFLAM, Novartis); lumiracoxib (COX-189; Novartis); D 1367 (Celltech Chiroscience, plc); R 807 (3 benzoyldifluoromethane sulfonanilide, diflumidone); JTE-522 (Japan Tobacco, Japan); FK-3311 (4′-Acetyl-2′(2,4-difluorophenoxy)methanesulfonanilide), FK 867, FR 140423, end FR 115068 (all Fujisawa, Japan); GR 253035 (Glaxo Wellcome); RWJ 63556 (Johnson & Johnson); RWJ 20485 (Johnson & Johnson); ZK 38997 (Schering); S 2474 ((E)-(5)-(3,5-di-tert butyl-4-hydroxybenzylidene)-2-ethyl-1,2-isothiazolidine-1,1-dioxide indomethacin; I Shionogi & Co., Ltd., Japan); zomepirac analogs, such as RS 57067 and RS 104897 (Hoffmann La Roche); RS 104894 (Hoffmann La Roche); SC 41930 (Monsanto); pranlukast (SB 205312, Ono-1078, ONON, ULTAIR@; SmithKline Beecham); SB 209670 (SmithKline Beecham); and APHS (heptinylsulfide).

A description of these classes of drugs and diagnostic agents and a listing of species within each class can be found, for instance, in Martindale, The Extra Pharmacopoeia, Twenty-ninth Edition (The Pharmaceutical Press, London, 1989), which is incorporated herein by reference in its entirety. The drugs or diagnostic agents are commercially available and/or can be prepared by techniques known in the art.

Poorly water soluble drugs which may be suitably used in the practice of the subject invention include but are not limited to alprazolam, amiodarone, amlodipine, astemizole, atenolol, azathioprine, azelatine, beclomethasone, budesonide, buprenorphine, butalbital, carbamazepine, carbidopa, cefotaxime, cephalexin, cholestyramine, ciprofloxacin, cisapride, cisplatin, clarithromycin, clonazepam, clozapine, cyclosporin, diazepam, diclofenac sodium, digoxin, dipyndamole, divalproex, dobutamine, doxazosin, enalapril, estradiol, etodolac, etoposide, famotidine, felodipine, fentanyl citrate, fexofenadine, finasteride, fluconazole, flunisolide, flurbiprofen, fluvoxamine, furosemide, glipizide, gliburide, ibuprofen, isosorbide dinitrate, isotretinoin, isradipine, itraconazole, ketoconazole, ketoprofen, lamotrigine, lansoprazole, loperamide, loratadine, lorazepam, lovastatin, medroxyprogesterone, mefenamic acid, methylprednisolone, midazolam, mometasone, nabumetone, naproxen, nicergoline, nifedipine, norfloxacin, omeprazole, paclitaxel, phenyloin, piroxicam, quinapril, ramipril, risperidone, sertraline, simvastatin, sulindac, terbinafine, terfenadine, triamcinolone, valproic acid, zolpidem, or pharmaceutically acceptable salts of any of the above-mentioned drugs.

Diagnostic agents can also be delivered use of the delivery particle of the invention. Diagnostic agents may be administered alone or combination with one or more drugs as described above. The diagnostic agent can be labeled by various techniques. The diagnostic agent may be a radiolabelled compound, fluorescently labeled compound, enzymatically labeled compound and/or include magnetic compound or other materials that can be detected using techniques such as X-ray, ultrasound, magnetic resonance imaging (MRI), computed tomography (CT), or fluoroscopy.

According to one preferred embodiment the active agent to be delivered by the delivery system of the invention is a cytotoxic drug (anti-tumor agents). Cytotoxic agents exemplified herein are docetaxel, paclitaxel and paclitaxel palmitate. Specific cytotoxic agent is docetaxel (DCTX), which is known to be a preferred drug of choice for treating hormone refractory prostate cancer (HRPC).

It is appreciated that in some cases the delivery particle may comprise more than one active agent. Further, the particle may be loaded with an active agent and a suitable adjuvant therefore, i.e. an ingredient that facilitates or modified the action of the principle active agent. For example, in immunotherapy, the adjuvant will be a substance included in a vaccine formulation to enhance or modify the immune-stimulating properties of a vaccine. According to another example, the particle may comprise a combination of a drug with a multi-drug resistant (MDR) inhibitor agent to potentiate the drug action; such combination may include Verapamil known to inhibit MDR to e.g. cyclosporine A (CsA).

Further, it may occur that the targeting agent has also a therapeutic or diagnostic benefit. Thus, according to some embodiments, the particle may include only the targeting agent as the principle active agent, or in addition to the targeting agent an active agent embedded in the particle's matrix or core. Examples where the targeting agent may serves also as the active principle is trastuzumab, which is also specifically exemplified hereinbelow.

The immunonanoparticles of the present invention are advantageous since they are capable of selectively binding to specific receptors or antigens and release the active agent at the desired site. The binding of the targeting agent to specific receptors or antigens triggers the transfer of the nanoparticles across biological barriers using endogeneous receptor mediated transcytosis and endocytosis systems. This will improve the therapeutic efficacy of the immunoparticles preparation when absent of the targeting agent as well as reduce adverse side effects associated with the active agent.

Nanoparticles undergo rapid clearance following IV administration by the reticuloendothelial system (RES). In order to inhibit the uptake of the nanoparticles by the RES, the nanoparticles may be modified at their surface with a hydrophilic polymer. The attachment of the hydrophilic polymer to the polymer forming the particle may be a covalent or non-covalent attachment, however, is preferably via the formation of a covalent bond to a linker anchored in the surface of the particle. The linker may be the same or different from the linker to which the targeting agent is bound. The outermost surface coating of hydrophilic polymer chains is effective to provide a particle with a long blood circulation lifetime in vivo.

According to one embodiment, the hydrophilic polymer is bound to a lipid, thus forming a lipopolymer, where the lipid portion anchors in the particle's surface.

The delivery system of the invention may be utilized for therapy or diagnosis, i.e. for targeted delivery of an active principle to a target site (cell or tissue). Thus, the invention also provides a pharmaceutical composition comprising the delivery system of the invention. According to one embodiment, the pharmaceutical composition is for the treatment or prevention of a disease or disorder, the delivery system being combined with physiologically and a pharmaceutically acceptable carrier.

The term “treatment or prevention” as used herein denotes the administering of a an amount of the active agent within the delivery system effective to ameliorate undesired symptoms associated with a disease, to prevent the manifestation of such symptoms before they occur, to slow down the progression of the disease, slow down the deterioration of symptoms, to enhance the onset of remission period of a disease, slow down the irreversible damage caused in a progressive chronic stage of a disease, to delay the onset of said progressive stage, to lessen the severity or cure a disease, to improve survival rate or more rapid recovery, or to prevent a disease form occurring or a combination of two or more of the above.

The term “effective amount” in accordance with this embodiment is an amount of the active agent embedded in the delivery particle in a given therapeutic regimen which is sufficient to treat a disease or disorder. For example, when treating cancer, the amount of the active agent, e.g. cytotoxic drug, is an amount of drug loaded delivery particles which will result, for example, in the arrest of growth of the primary tumor, in a decrease in the rate of occurrence of metastatic tumors, or a decrease in the number of metastatic tumors appearing in the individual or in a decrease in the rate of cancer related mortality. Alternatively, when the drug loaded delivery system is administered for cancer prevention, an effective amount will be an amount of said particles which is sufficient to inhibit or reduce the occurrence of primary tumors in the treated individual. The pharmaceutically “effectiize amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art. For example, the amount may depend on the type, age, sex, height and weight of the patient to be treated, the condition to be treated, progression or remission of the condition, route of administration and the type of active agent being delivered.

The effective amount is typically determined in appropriately designed clinical trials (dose range studies) and the person versed in the art will know how to properly conduct such trials in order to determine the effective amount. As generally known, an effective amount depends on a variety of factors including the mode of administration, type of polymer and other components forming the nanoparticle, the reactivity of the active agent, the type and affinity of the targeting agent to its corresponding binding member, the delivery systems' distribution profile within the body, a variety of pharmacological parameters such as half life of the active agent in the body after being released from the nanoparticle, on undesired side effects, if any, on factors such as age and gender of the treated subject, etc.

In this case, for treatment purposes the drug loaded delivery particles of the invention may be administered over an extended period of time in a single daily dose (e.g. to produce a cumulative effective amount), in several doses a day, as a single dose for several days, etc. so as to prevent the damage to the nervous system.

As indicated above, the nanoparticles according to the present invention may be administered in conjunction with one or more pharmaceutically acceptable carriers. The properties and choice of carrier will be determined in part by the particular active agent, the particular nanoparticle, as well as by the particular method used to administer the composition. Accordingly, there is a wide variety of suitable formulations of the delivery system of the present invention, including, without being limited thereto, oral, intranasal, parenteral (subcutaneous, intravenous, intramuscular, interperitoneal), rectal, pulmonary (e.g. by inhalation) and vaginal administration. Preferably the route of administration of the delivery system of the invention is parenteral.

Formulations suitable for parenteral administration include, without being limited thereto, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. The nanoparticles can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

A person skilled in the art would readily be able to determine the appropriate concentrations of the active agent, amounts and routes of administration to deliver an efficacious dosage of the active agent over time. Furthermore, one skilled in the art may determine treatment regimens and appropriate dosage using the nanoparticles of the present invention, inter alia, depending upon the level of control over release of the entrapped or encapsulated active agent.

Considering the above, the invention also provides a method for treating a disease or disorder comprising administering to a subject in need an effective amount of the drug-loaded delivery system of the invention.

The types of conditions which may be treated with the delivery system of the invention are numerous, as appreciated by those versed in the art. A non-limiting list of conditions include cancer, conditions associated with the inflammatory states (inflammation or auto-immune conditions) such as rheumatoid arthritis, neurodegenerative disorders, infections, endocrine disorders (e.g. primary or secondary adrenocortical insufficiency; congenital adrenal hyperplasia, hypercalcemia associated with cancer, non-suppurative thyroiditis); collagen diseases (e.g. pemphigus bullous dermatitis, severe erythema, multi-herpetiformis forme (Stevens-severe seborrheic Johnson syndrome), dermatitis, exfoliative dermatitis, Severe psoriasis, mycosis fungoides); dermatologic diseases, allergic states (e.g. bronchial asthma, drug hypersensitivity, contact dermatitis reactions, atopic dermatitis, urticarial transfusion, serum sickness reactions, seasonal or perennial, acute noninfectious allergic rhinitis laryngeal edema); ophthalmic diseases (e.g. severe acute and chronic allergic and inflammatory processes involving the eye, such as: herpes zoster ophthalmicus, sympathetic ophthalmia iritis, iridocyclitis, anterior segment chorioretinitis inflammation, diffuse posterior uveitis, allergic conjunctivitis and choroiditis, allergic corneal marginal optic neuritis ulcers, keratitis); respiratory diseases (symptomatic sarcoidosis, loeffler's syndrome, aspiration pneumonitis, tuberculosis); hematologic disorders (e.g. acquired (autoimmune) hemolytic anemia, idiopathic thrombocytopenic purpura, secondary thrombocytopenia, erythroblastopenia (RBC anemia). congenital (erythroid) hypoplastic anemia); and edematous states; neoplastic diseases; and pathological conditions of the nervous system (e.g. multiple sclerosis).

In accordance with one embodiment, the invention provides a method for the treatment of cancer, by targeting, by appropriate MAbs the delivery system loaded with an anti-cancer drug (e.g. docetaxel and paclitaxel palmitate) to target cells.

The present invention additionally relates to a method of imaging in a subject's body a target cell or target tissue, the method comprising:

(a) providing said subject with a delivery system of the invention carrying a contrasting agent, wherein the nanoparticles are associated with one or more targeting agents effective to target said delivery system to said target cell or target tissue;

(b) imaging said contrasting agent in said body.

As indicated above, the delivery system of the invention may comprise a combination of a contrasting agent (imaging agent) and a therapeutic agent. Thus, by the use of the targeting system of the invention, a dual effect may be achieved, whereby the delivery of a drug may also be imaged.

The delivery device of the invention loaded with a contrasting agent may be utilized in different imaging techniques typically employed in medical diagnostics. Such include, without being limited thereto, X-ray (computer tomography (CT) of CAT scan), ultrasound, 7-scintigraphy or MRI imaging.

The contrasting agent may be any agent known in the art of imaging. An example includes, without being limited thereto, coumarin-6, gadolinium derivates iodized oils such as lipiodol (ethyl ester of fatty acids of poppyseed oil with iodine concentration of 38%), non ionic contrast medium such as iopromide, iopamidol.

As appreciated, while the invention is described in this detailed description with reference to pharmaceutical and diagnostic compositions, it is to be understood that also encompassed within the present invention is the use of the delivery system for other applications and in other forms.

As used in the specification and claims, the forms “a”, “an” and “the” include singular as well as plural references unless the context clearly dictates otherwise. For example, the term “an antibody” includes one or more different antibodies and the term “a contrasting agent” includes one or more contrasting agents.

Further, as used herein, the term “comprising” is intended to mean that the delivery system include the recited elements, but not excluding others. The term “consisting essentially of” is used to define the delivery system that include the recited elements but exclude other elements that may have an essential significance on the treatment or imaging procedure. “Consisting of” shall thus mean excluding more than trace elements of other elements. Embodiments defined by each of these transition terms are within the scope of this invention.

Further, all numerical values, e.g. when referring the amounts or ranges of the elements constituting the device's layers, are approximations which are varied (+) or (−) by up to 20%, at times by up to 10% of from the stated values. It is to be understood, even if not always explicitly stated that all numerical designations are preceded by the term “about”.

### DESCRIPTION OF SPECIFIC EXAMPLES

### Example 1

### Cross-Linker (OMCCA) Synthesis

For the synthesis of Octadecyl-4-(maleimidomethyl)cyclohexane-carboxylic amide (OMCCA), 100 mg of Sulfosuccinimidyl-4-(N-maleimidomethyl)cyclohexane-1-carboxylate (SMCC Pierce, Ill., USA) and 80 mg of stearylamine (SA, Sigma Chemical, MO, USA) were dissolved in 8 ml chloroform and in 41 ul of triethylamine (Reidel-de-Haen, Sigma-Aldrich Chemie GmbH, Steinheim, Germany and the reaction was incubated at 50° C. for 4 hours. The solution was washed three times with 1% HCl and the chloroform was evaporated under reduced pressure. The product was desiccated overnight and weighted. The yield was about 90% and linker formation was confirmed by H-NMR (Mercury VX 300, Varian, Inc., CA, USA), IR (Vector 22, Bruker Optics Inc, MA, USA) and LC-MS (Finnigan LCQDuo, ThermoQuest, NY, USA).

### H-NMR, IR and LC-MS Analysis

H-NMR (of OMCCA in CDCl₃): Peaks at: 0.008, 0.849, 0.0893, 1.009, 1.245, 1.450, 1.577, 2.157, 2.160, 2.167, 2.173, 2.178, 2.181, 3.349, 3.372, 6.692, 7.257 ppm

IR: Peaks at: 626.89, 695.63, 722.35, 834.46, 899.52, 910.59, 934.79, 1045.94, 1120.05, 1163.30, 1214.60, 1260.82, 1362.15, 1408.40, 1431.38, 1468.04, 1541.02, 1629.86, 1701.35, 2850.80, 2923.84, 3087.43, 3318.81, 3453.91 cm⁻¹

LC-MS: Peak at: 490.17, 491.26

The analysis of the NMR and IR spectrum confirms the formation of the linker OMCCA, while the LC-MS spectra clearly corroborates the molecular weight of the product which is 490 g/mol.

### Example 2

### Polymers Syntheses

### (A) PEG-PLA Synthesis and Characterization

PEG-PLA (5:20) was synthesized according to well known procedure as described by Bazile D. et al. [Bazile D, et al. J Pharm Sci, 84: 493-498 (1995)]. In brief, 2 g of methoxy polyethylene glycol mw 5000 (Sigma-Aldrich Chemie GmbH, Steinheim, Germany) were mixed with 12 g of D, L-lactide (Purasorb, Purac, Gorinchem The Netherlands) for 2 hours under dried conditions at 135° C.

The polymer was analyzed by H-NMR (Mercury VX 300, Varian, Inc., CA, USA) and by differential scanning calorimetry (STARe, Mettler Toledo, Ohio, USA).

Diblock polyethylene glycol (mw 5000) and polylactide (mw 20000) polymer (PEG-PLA 5:20) was synthesized as described above. Gel permeation chromatography (GPC) exhibited mw of 20000 and polydispersity index [PD.I] of 1.47. The polymer was analyzed by H-NMR and by differential scanning calorimetry (DSC).

### H-NMR and DSC Analysis

¹H-NMR (of PEG-PLA (5:20)): Peaks at: −0.010, −0.008, −0.001, 1.206, 1.543, 1.560, 1.567, 1.581, 1.591, 3.641, 5.136, 5.145, 5.159, 5.169, 5.182, 5.192, 5.207, 5.215, 5.231, 7.256

DSC (PEG-PLA (5:20) 3.98 mg):

Peak1: integral −118.88 mJ, onset 28.70° C., peak 43.24° C., heating rate 10° C./min

Peak2: integral −1234.12 mJ, onset 237.54° C., peak 273.98° C., heating rate 10° C./min

The analysis of the NMR and DSC spectrum clearly show the formation of the diblock polymer. It can be deduced that PEG is attached covalently to PLA.

(B) Polylactide and poly(ethylene glycol-co-lactide) Synthesis

The polymers: polylactide (PLA) and poly(ethylene glycol-co-lactide) (mPEG-PLA) were synthesized using the ring opening polymerization method in the presence of stannous 1-ethylhexanoate as catalyst (4). In case of synthesis of PLA; D,L-lactide (30 g) and benzyl alcohol (32 mg) as co-catalyst, are dissolved in 250 ml of dried toluene while in the case of synthesis of mPEG-PLA; 1.5 g of methoxy polyethylene glycol (mPEG, MW 5000) was used as co-catalyst and added to 250 ml of dried toluene containing already 30 g of D,L-lactide. The refluxing mixture was stirred over a Dean-Stark apparatus over a period of 4 h for azeotropic removal of water. Stannous 1-ethylhexanoate (245 mg) was added following the removal of the remaining water. Then, the mixture was heated to 135° C. for 4 h. The crude polymers were dissolved in methylene chloride and precipitated twice into 4 liters of cold propyl ether/petroleum ether mixture (3:2). Prior to characterization the polymers were vacuum dried. The synthesis of the co-polymer is depicted in the following Scheme 2:

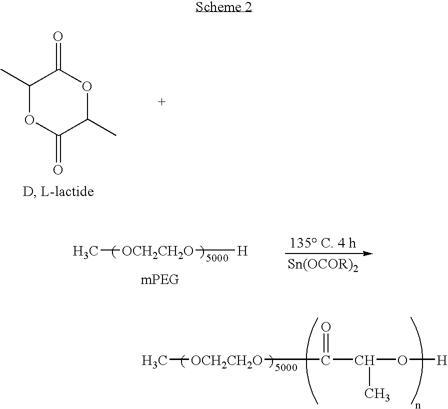

Polylactide and Poly(ethylene glycol-co-lactide) Characterization

The co-polymers were characterized by gel permeation chromatography (GPC) system consisting of a Waters 1515 Isocratic high performance liquid chromatography (HPLC) pump, with 2410 refractive index detector (Waters, Milford, Mass.) and a Rheodyne (Cotati, Calif.) injection valve with a 20 μl loop. Samples were eluted with chloroform though a linear Styrogel HR column, (Waters, Mass.), at a flow rate of 1 mL/min. The molecular weights were determined relative to polystyrene standards (Polyscience, Warrington, Pa.) with a molecular weight range of 54-277.7 KDa using BREEZE 3.20 version (copyright 2000, Waters Corporation computer program). Thermal analysis was determined on a Mettler TA 4000-DSC differential scanning calorimeter (Mettler-Toledo, Schwerzzenbach, Switzerland), calibrated with Zn and In standards, at a heating rate of 20° C./min under nitrogen atmosphere. ¹H-NMR spectra (in CDCl₃) were recorded on Varian 300 MHz spectrometers using TMS as internal standard (Varian Inc., Palo Alto, Calif., USA).

Polymers with molecular weights in the range of 20 000-146 000 were obtained. The basic chemical structure of PLA and mPEG-PLA polymers was confirmed by ¹H-NMR spectra which fit their composition. Overlapping doublets at 1.55 ppm are attributed to the methyl groups of the D- and L-lactic acid repeat units. The multiplets at 5.2 ppm correspond to the lactic acids CH group. When mPEG-PLA spectra is analyzed a peak at 3.65 ppm was detected which fits the methylene groups of the mPEG.

According to the data obtained from the thermographs (see Table 1), only the PEG:PLA₂₀ exhibited crystalline domains with the appearance of a melting point thermal event at 43.2° C. The observed crystalline domains are probably associated with the marked presence of the crystalline PEG₅₀₀₀ in the mPEG-PLA₂₀₀₀₀ co-polymer chain as suggested by the lack of melting point event in the thermographs of PLA₄₀₀₀₀, mPEG-PLA₁₀₀₀₀₀ and PLA₁₀₀₀₀₀ which show only a glass transition temperature, T_{g} (see Table 1). Indeed T_{g} increases with increase of PLA chains from 40000 to 100000 as noted in Table 1. It is well known that mPEG chains which are highly ordered elicit a crystalline character while PLA chains are less ordered exhibiting an amorphous state. This increase in PLA chains in the MPEG-PLA on the expense of PEG will increase the amorphous character of the co-polymers and consequently T_{g} will increase.

TABLE 1
Physical properties of synthesized polymers

| | Molecular weight^{a} | | | |
|---|---|---|---|---|
| Polymer | Mn^{c} | Mw^{c} | T_{g} (° C.)^{b} | Tₘ (° C.)^{b} |
| PEG:PLA₂₀ | 20000 | 29000 | — | 43.2 |
| PEG:PLA₄₀ | 37000 | 52000 | 34.1 | — |
| PEG:PLA₁₀₀ | 87000 | 136000 | −19.4; 49.1 | — |
| PLA₁₀₀ | 80000 | 83000 | 65.3 | — |

^{a}molecular weight determined by GPC.
^{b}glass transition temperature (T_{g}) and melting point (Tₘ) determined by DSC.
^{c}Mn is the number average of the molecular weight and Mw is the weight average of the molecular weight.

### Example 3

### (A) Nanoparticles (NPs) Preparation and Characterization

### NP's Preparation

The PLA nanoparticles were prepare by the nanoparticles-polymer interfacial deposition method as described by Fessi H et al. [Fessi H, et al. Int. J. Pharm. 55: R1-R4 (1989)]. In brief, 88 mg of the polymer PLA (polylactide, 30 KDa purchased from Boehringer Ingelheim) and 38 mg of the co-polymer PEG-PLA, 5:20 (polyethylene glycol of MW of 5000 and polylactide MW of 20,000) were dissolved in 20 ml acetone, a water-miscible organic solvent. To this organic phase 10 mg of the drug docetaxel were added. For coupling of an antibody, to the organic phase, 20 mg of the linker OMCCA were added. The resulting organic phase was then added to 50 ml of aqueous phase which contained 100 mg Solutol® HS15 (BASF, Ludwigshafen, Germany), as a surfactant (Macrogol 15 hydroxystearate). The dispersion was mixed at 900 rpm over 1 hr and then evaporated under reduced pressure to 20 ml. the NPs were washed with Phosphate Buffered Saline (PBS) 5-6 times using vivaspin 300 KDa cut-off. Spherical polymeric, nanometric (100-500 nm) particles were spontaneously formed under these conditions.

TABLE 2
Linker (OMCCA) containing formulation

| Organic phase | Aqueous phase |
|---|---|
| PEG (MW 5000)-polylactide | Solutol ® HS 15^{(a)} 0.5% w/v |
| (MW 20,000) [PEG-PLA 5:20] 0.88% | Water 50 ml |
| polylactide (MW 30,000) | |
| [PLA 30] 0.19% | |
| Acetone 20 ml | |
| OMCCA 0.1% w/v | |

^{(a)}Solutol^{R} HS 15 (0.5% w/v): Macrogol 15 hydroxystearate was dissolved in water at a concentration of 0.5%.

### Drug Incorporation Efficacy

Drug encapsulation (incorporation) efficacy was determined using HPLC system consisting of Kontron instruments (Watford, UK) 325 pump, Kontron instruments 332 detector adjusted at 227 nm and Kontron instruments 360 autosampler. Separation was achieved by LichroCART (Merck Darmstadt, Germany) C18 (250*4 mm, 5 um) column. The mobile phase was 50% acetonitrile in water at flow rate of 1 ml/min. the retention time of docetaxel was 10 minutes.

### Nanoparticle Characterization

### (1) Particle Size Analysis

Mean diameter and particle size distribution measurements were carried out utilizing an ALV Noninvasive Back Scattering High Performance Particle Sizer (ALV-NIBS HPPS, Langen, Germany) at 25° C. and using water (refractive index: 1.332; viscosity: 0.894543) as the diluent. A laser beam at 632 nm wavelength was used. The sensitivity range was 0.5 nm to 5 μm.

### (2) Zeta Potential Measurements

The zeta potential of the NPs/immunoNPs was measured using the Malvern zetasizer (Malvern, UK) diluted in double distilled water.

### (3) Morphological Evaluation Using TEM

Morphological evaluation for the immunoNPs was performed by means of transmission electron microscopy (TEM) using gold labeled goat anti-human IgG (Jackson ImmunoResearch Laboratories, PA, USA).

Blank trastuzumab immunoNPs (containing no active ingredient) were incubated with a gold labeled anti-human IgG and negatively stained with phosphotungstic acid (PTA) 2% pH 6.4.

### Results

### Drug Incorporation Efficiency

The encapsulation efficiency of the cytotoxic drug docetaxel (DCTX) in the nanoparticles and in the immunonanoparticles was determined by HPLC and found to be 100% and 49%, respectively. It was interesting to note that the theoretical drug content of the DCTX loaded NPs, 7.4%. w/w (initial weight ratio PLA: PEG-PLA: DCTX; 88:38:10) was significantly higher than the drug content of DCTX immunonanoparticles, 3.3%, w/w (initial weight ratio PLA: PEG-PLA: DCTX: OMCCA; 88:38:10:20). This marked difference in DCTX content may be attributed to the presence of the linker in the polymeric matrix. During nanoparticle formation, the linker probably competes with DCTX and reduce its incorporation extent from 7.4 to 3.3%.

### Particle Size Analysis

The average and particle size distribution of the various NPs was measured using the ALV method. It was observed that the mean diameter of the blank NPs (containing no active ingredient) was 60 nm while the diameter was 150 and 180 nm for the blank immunoNPs (containing no active ingredient) and for DCTX loaded immunoNPs, respectively. The marked increase in diameter of the NPs should be related to the linker's presence which probably decreases the acetone diffusion towards the aqueous phase allowing the formation of larger NPs.

### Zeta Potential Measurements

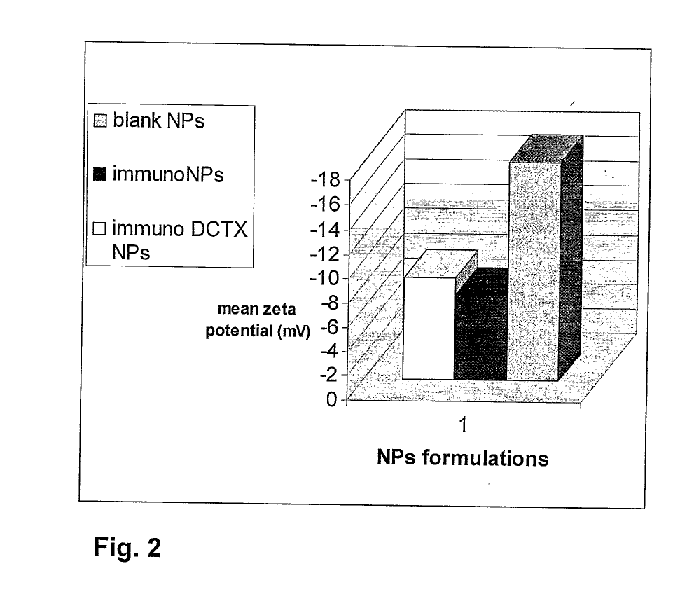

The zeta potential of the blank NPs was −18 mV and decreased to −7 mV for the antibody conjugates NPs (FIG. 2). The decrease in zeta potential should be attributed to the positive charge of trastuzumab at pH 7.4 since its isoelectric point is 9.

### Morphological Evaluation Using TEM

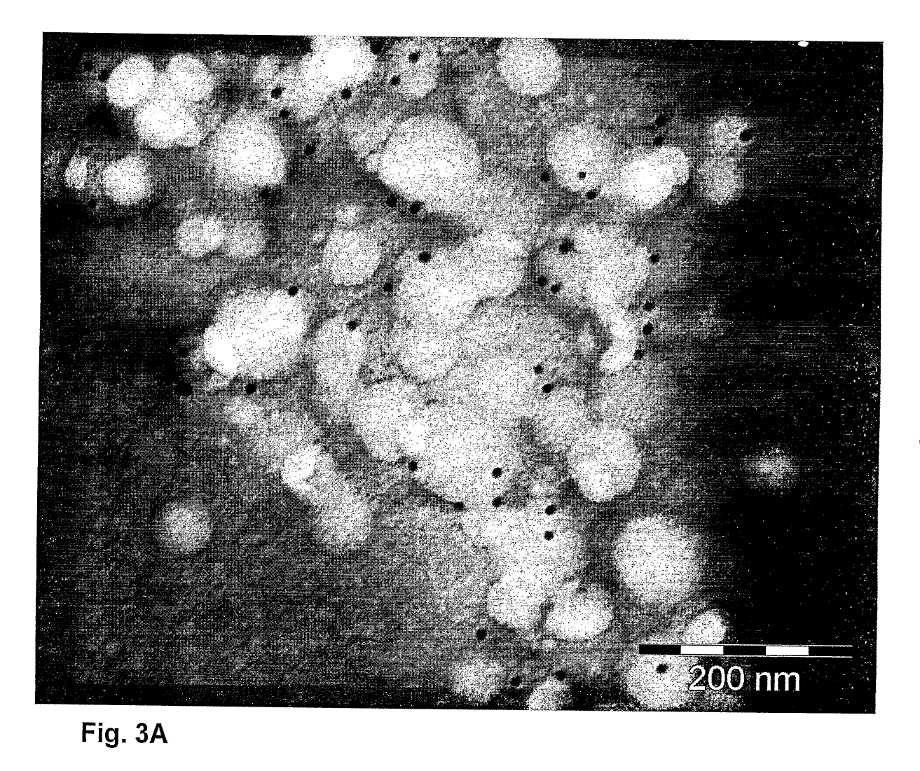

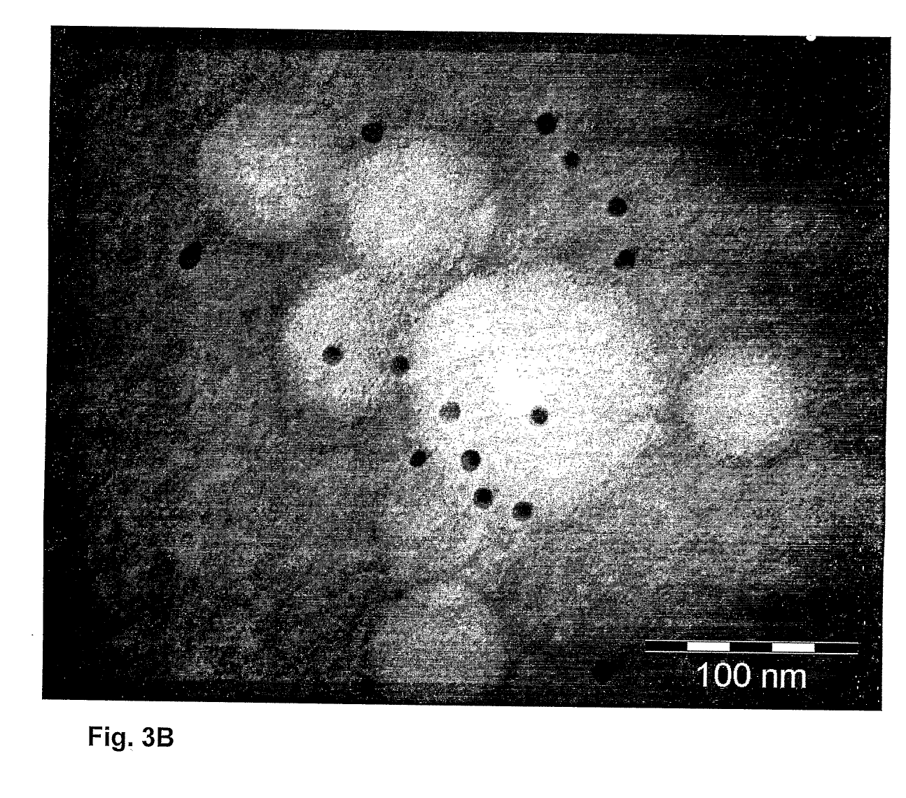

It can be noted from the results depicted in FIGS. 3A-3B that each gold black spot represents one trastuzumab molecule attached to the nanoparticle surface. It can be deduced that the MAb has been efficiently conjugated to the surface of the nanoparticle by the linker and the reaction conditions did not affect the initial affinity of the MAb to the secondary antibody

(B) Conjugation with Targeting Moiety

### Antibody Modification for Thiol Groups Generation

Increment of thiol groups on the MAb was preformed using the 2-iminothiolane reagent [Traut's reagent, Sigma-Aldrich Chemie GmbH, Steinheim, Germany, Traut R R, et al. Biochemistry. 12(17):3266-73 (1973); Jue R, et al. Biochemistry. 17(25):5399-406 (1978)]. Traut reagent was incubated for 45 min with purified trastuzumab at molar ratio of 30:1, respectively. The Traut modified MAb was separated on HiTrap desalting column (Amersham Bioscience, Uppsala, Sweden). Fractions containing the modified MAb were determined by UV at 280 nm. Free thiol groups were determined with 5,5′-dithio-bis(2-nitrobenzoic acid) (Ellman's reagent, Sigma-Aldrich Chemie GmbH, Steinheim, Germany), by monitoring the change in absorbance at 412 nm. Once reacted with Traut's reagent, mAb possess reactive sulfhydryls that can be used in conjugation protocols with sulfhydryl-reactive cross-linking reagents bearing a maleimide group such as OMCCA. The following Scheme (3) illustrates a possible conjugation reaction between reduced antibody and maleimide group of the linker:

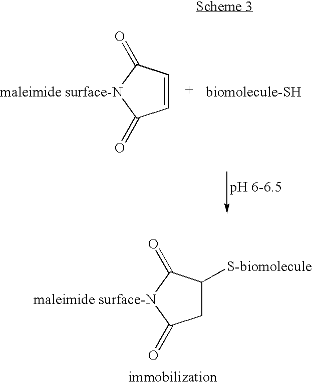

### Coupling Reaction

Freshly prepared nanoparticles consisting of 88 mg of the polymer PLA (polylactide, 30 KDm 38 mg of the co-polymer PEG-PLA, 5:20, 0 or 10 mg of the drug docetaxel and 20 mg of the cross-linker OMCCA equivalent to an overall amount of blank nanoparticles of 146 mg or 156 mg of DCTX nanoparticles (PLA: PEG-PLA: DCTX: OMCCA; 88:38:0/10:20) were adjusted to pH 6.5 with 0.1N NaOH and incubated with Traut modified trastuzumab (final concentration 1 mg/ml) overnight at 4° C. under continuous agitation and under nitrogen atmosphere. Unreacted maleimide groups were blocked through incubation with 2-mercaptoethanol (Pierce, Ill., USA) for 30 min. Unconjugated antibody and 2-mercaptoethanol were separated from immunonanoparticles by gel filtration over a Sepharose CL-4B column (Amersham Bioscience, Uppsala, Sweden). Coupling efficiency was evaluated by the BCA protein assay (Bicinchoninic Acid protein assay) (Pierce, Ill., USA) as described [Smith P. K., et al. Anal. Biochem. 150:76-85 (1985)].

For preparation of immunonanoparticles with various amounts of conjugated antibody, the initial ratio of Traut modified trastuzumab to maleimide-activated particles was varied. The actual investigated ratio was 146 mg of blank NPs or 156 mg of DCTX NPS for 26 mg of MAb.

Morphological evaluation for the final immunonanoparticles was performed by means of transmission electron microscopy (TEM) using gold labeled goat anti-human IgG (Jackson ImmunoResearch Laboratories, PA, USA).

### Drug Content Determination

The final drug content in the nanoparticles was evaluated as follows: the colloidal dispersion comprising a final volume of 20 ml is first ultrafiltrated using Vivaspin of 30000 daltons cutoff (Sartorius, Goettingen, Germany) to obtain 2-3 ml of clear ultrafiltrate. The concentration of DCTX in the ultrafiltrate is measured by HPLC. The remaining total volume of colloidal dispersion is then lyophilized, weighted and subjected to total DCTX content analysis using HPLC for final calculation of drug content in the nanoparticles. Various initial increasing drug ratios will be tested to identify the optimal formulation. Furthermore, the presence of possible tiny drug crystals in the colloidal dispersion will be also monitored.

### Absorption of Trastuzumab to Blank Nanoparticles

The purpose of this determination was to evaluate whether trastuzumab molecules are physically absorbed onto blank nanoparticles, i.e. nanoparticles containing no linker anchored at their surface. To this end, 100 ul (1 mg) of trastuzumab 7.5 mg/ml solution were mixed over 1 hour at room temperature with 1 ml of blank positive and negative charged nanoparticle aqueous dispersions containing a total amount of 125 mg nanoparticles. The mixture (750 ul of) was then washed 5 times with 30 ml of PBS and the diluted dispersion was filtered through vivaspin 300 KDa cut-off using centrifugation (4000 rpm, 30 min) to remove unabsorbed MAb molecules.

The protein concentration was determined using PCA protein assay to detect the presence of MAb molecules in the nanoparticle supernatant.

### Results

### SH Group Determination

The number of sulfhydryl groups on the modified MAb was determined using Ellman's reagent compared to cysteamine as standard. The intact trastuzumab and the Traut modified trastuzumab were diluted with PBS buffer containing 0.1M EDTA pH 8 and incubated with Ellman's reagent. The Traut modified trastuzumab SH groups per MAb was determined to be 31.5 as compared to 1.4 in the intact trastuzumab.

### Coupling Efficiency Determination

The amount of the MAb conjugated to the NPs was determined using BCA protein assay. NPs were degraded with 0.1N NaOH at 50° C. and incubated with assay reagent. The coupling efficiency for the immunoNPs (without the drug DCTX) and for the immuno DCTX loaded NPs was 71 and 77%, respectively.

### Absorption of Trastuzumab to Blank Nanoparticles

The ratio between the amount of trastuzumab before and after separation for the positive and negative formulations was 4.2 and 2.7%, respectively.

These results ensure that there was no absorption of MAb molecules onto the nanoparticles following successive washings with PBS and therefore the coupling of MAb to linker containing nanoparticles is most probably mediated by a covalent conjugation since all the successive washings and purification processes during immunonanoparticle preparation are carried out using PBS at similar dilution extent.

The lack of MAb adsorption on vivaspin membranes was validated in previous experiments when MAb aqueous solutions were subjected to identical experimental conditions and the concentrations of MAb in the supernatant and ultrafiltrate were found to be similar.

### (C) Cell Culture Studies

### HER-2 Over-Expression Determination

HER-2/neu over-expression was evaluated in breast cancer cell line: SK-BR-3 and in prostate cancer cell line: LNCaP. SK-BR-3 Cells were grown on cover slips to subconfluency. Cells were fixated using fresh 4% paraformaldehyde for 10 min than, cells were washed and self-fluorescence was blocked with 5% BSA. Cells were incubated with primary MAb, either intact or Traut modified (0.1 mg/ml, 0.05 mg/ml in 400 ul per well) overnight at 4° C. Cells were washed and incubated with a 1:50 dilution of FITC conjugated goat-anti human IgG (Jackson ImmunoResearch Laboratories, PA, USA) over 1 hour at room temperature. Secondary antibody were washed following mounting and then cells were taken for observation using either fluorescence microscope or confocal microscope (Zeiss, Axioveit 135M, Oberkochen, Germany).

LNCaP cells were trypsinized after reaching confluence and transferred into tubes (10⁶ cells per tube). Medium was discarded and fixation performed using fresh 4% paraformaldehyde for 10 min. Cells were washed and self-fluorescence was blocked with 5% BSA. Cells were washed and incubated with several dilutions of trastuzumab for 1 hour 4° C. Cells were washed and incubated with a 1:100 dilution of FITC conjugated goat-anti human IgG for 1 hour at room temperature. Secondary antibody were washed and analyzed by flow cytometry (FACScom, B&D)

### Results

### HER-2/Neu Over-Expression Determination

Immunostaining and FACS analysis for the determination of HER-2/neu over-expression in various cancer cell lines such as SK-BR-3 (breast cancer cells) and LNCaP (prostate cancer cells) were performed as described above. Fixed cells were incubated with trastuzumab in order to detect HER-2/neu over-expression. Cells which were not incubated with trastuzumab but with the secondary FITC conjugated goat anti human IgG were used as controls.

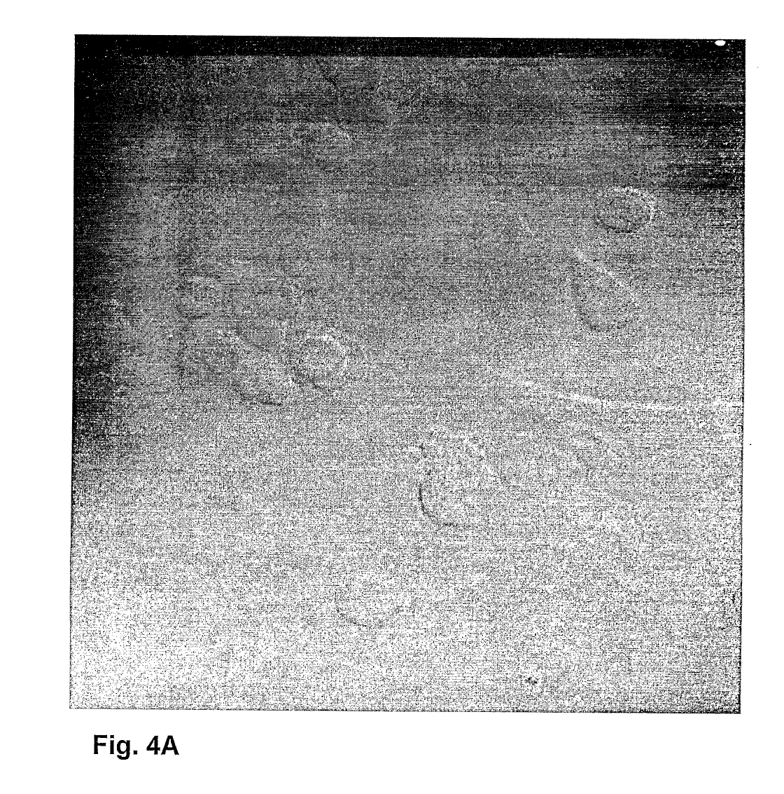

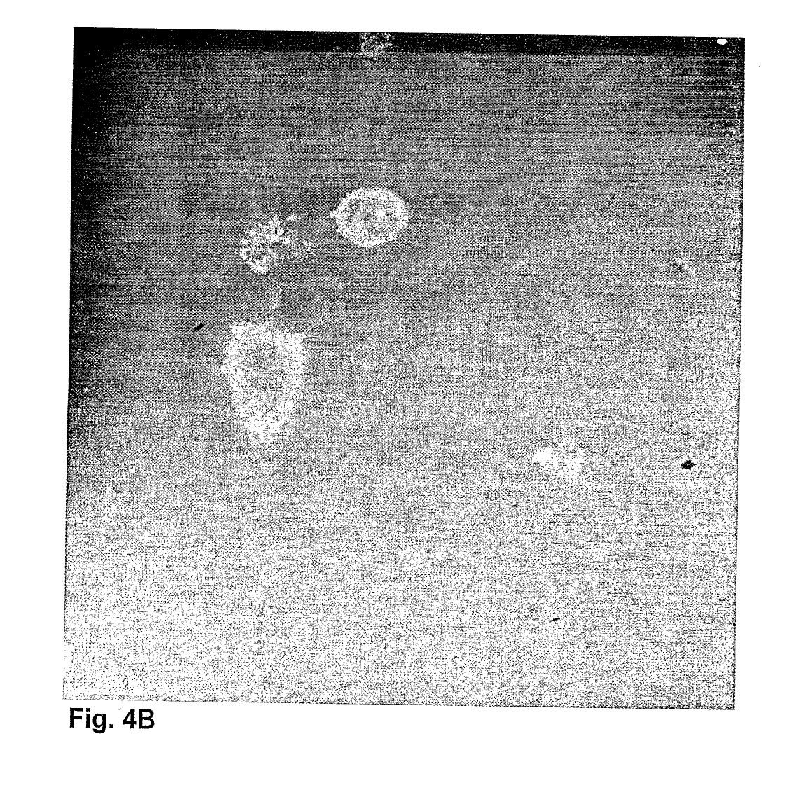

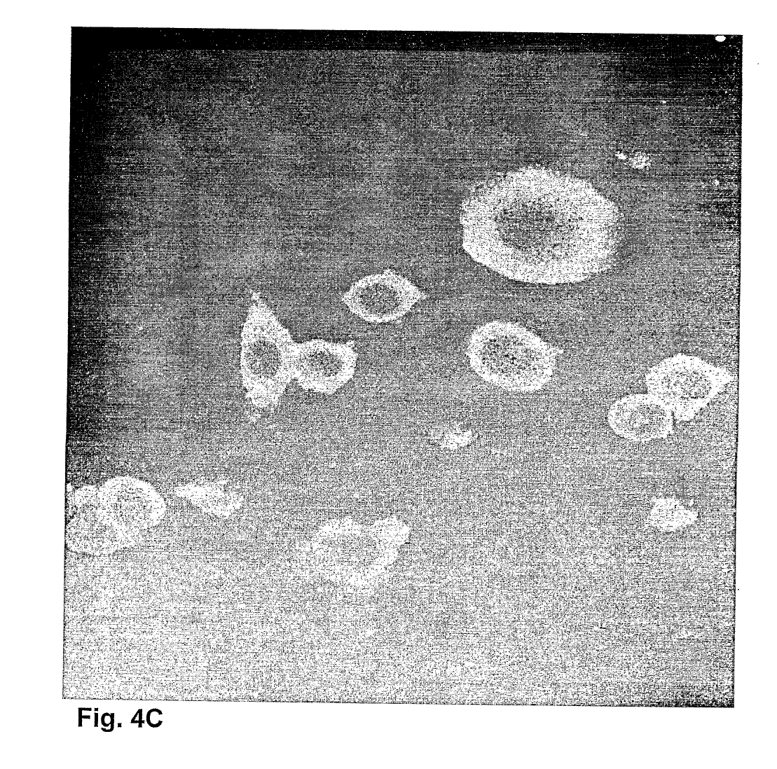

The confocal microphotographs show the affinity of intact and Taut modified trastuzumab to SK-BR-3 cells (FIGS. 4A-4C). It can be noted from FIG. 4A that there is no fluorescence in the absence of trastuzumab whereas in FIGS. 4B and 4C, a marked cell surface fluorescence is noted, clearly indicating the presence of HER-2 on the cell surface.

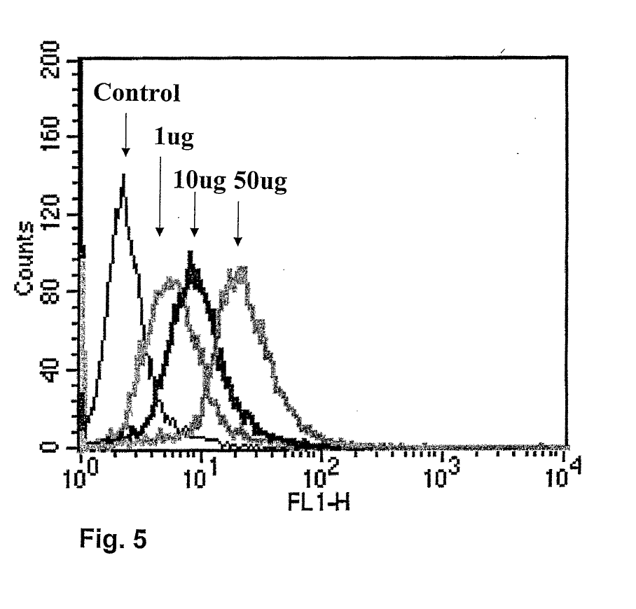

FACS analysis diagrams (FIG. 5) show increasing affinity of trastuzumab to LNCaP cells with increasing amounts of the MAb. The data clearly indicate that HER-2/neu is over-expressed on the membranes of the cells.

### In Vitro Binding Visualization

### Fluorescence and Confocal Laser Scanning Microscopy (CLSM) Analysis of Cellular Binding of Immunonanoparticles

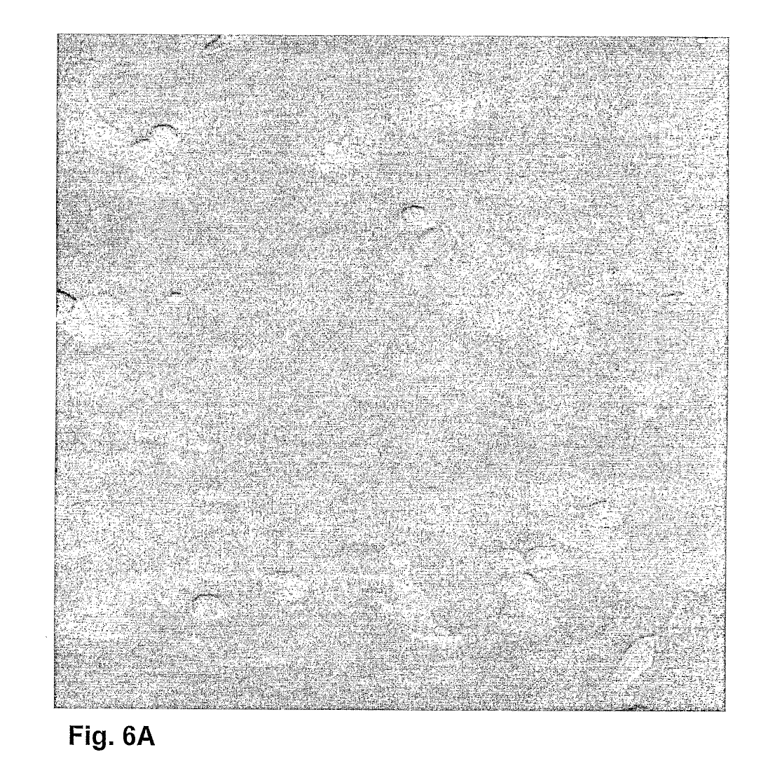

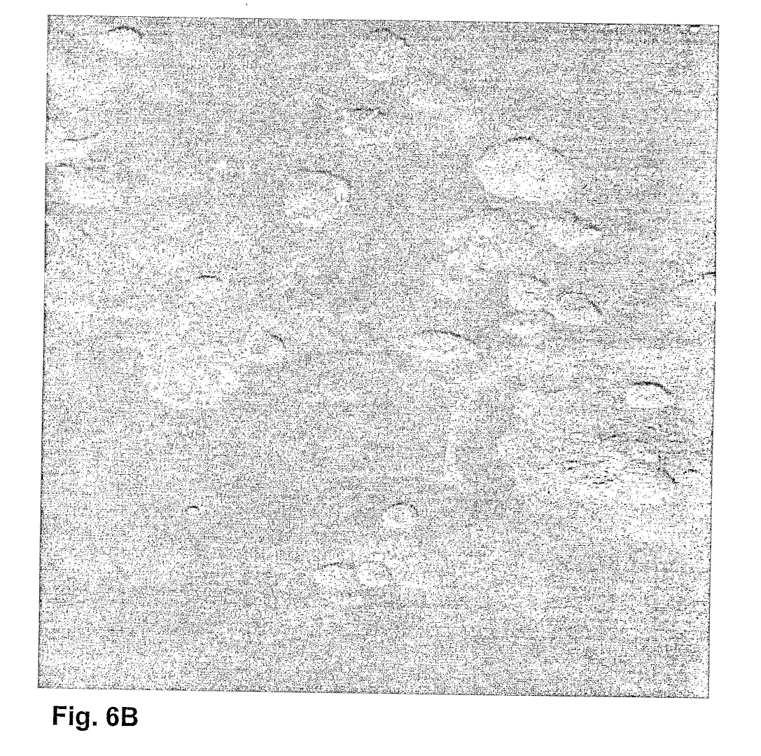

Fixed SK-BR-3 cells were incubated with trastuzumab conjugated nanoparticle formulations following incubation with FITC labeled goat anti-human IgG. Cells were examined using fluorescence (results not shown) and confocal microscopes (FIGS. 6A-6B). The binding of the immunoNPs to the cell surface was proportional to the concentration of cross-linker. The fluorescence elicited by the formulation composed of initial weight ratio PLA: OMCCA; 50:10 (FIG. 6B) was significantly higher than the fluorescence of the formulation composed of initial weight ratio PLA: OMCCA; 50:6, (FIG. 6A) owing to the MAb subsequent higher density i.e. the concentration of MAb in B is 150 ug/ml while it is only 40 ugml in A as determined by BCA assay.

SK-BR-3 and LNCaP cells were grown on cover slips to subconfluency. Cells were incubated with NPs in media at 4° C. for different time intervals, washed and incubated with a 1:100 dilution of FITC conjugated goat-anti human IgG for 1 hour at room temperature. Secondary antibody were washed following mounting in glycerol and observed with a fluorescence and confocal microscope.

The confocal microscopy is presented in FIGS. 6A-6B confirming that the binding to cells was much more significant with the formulation containing nanoparticles conjugated to trastuzumab with PLA/OMCCA ratio of 50:10 mg/mg linker (FIG. 6B) as compared to the same particles with PLA/OMCCA ratio of 50:6 mg/mg (FIG. 6A).

### (D) Variability of MAb

The aim of the study was to show that two different MAbs can be conjugated on the same nanoparticle. To this end, two different MAbs were used: trastuzumab and AMB8LK an anti H-ferritin monoclonal antibody (purchased from MAT, Evry, France). Each MAbs was marked differently with fluorescent probe.

### Sulforhodamine B Chloride Acid Labeling of Trastuzumab (Red Color)

Trastuzumab (21 mg in 1 ml) were washed with sodium bicarbonate 0.165M buffer pH 9.4. 100 μl of 1 mg/ml sulforhodamine B chloride acid in DMF solution were added gradually to the MAb solution while stirring. The reaction was incubated for 1 hr at 4° C. To separate labeled MAb from free sulforhodamine B chloride acid PD10 column was used and washed with PBS-EDTA pH 7.2 (1.8 g NaHPO3(60 mM), 4.35 g NaCl (150 mM), 0.93 g EDTA (5 Mm)).

Final volume of the collected labeled MAb was 1850 μl. 5 μl of the solution were diluted 1:200 with PBS-EDTA and the sample was read in UV spectrophotometer at 280 nm (protein) and at 570 nm (sulforhodamine B chloride acid).

1 mg/ml IgG-1.4 Aₚᵣₒₜₑᵢₙ

0.0464 mg/ml IgG-0.065 Aₚᵣₒₜₑᵢₙ

Degree of Labeling (DOL):

Aₘₐₓ*MW/[protein]*ε_{dye}=0.008*150000/0.04617*120000=0.2

Labeled MAb was concentrated to 1 ml in 30K filter eppendorf (Pall), than 18.4 mg in 876 μl incubated with 6 mg 2-mercaptoethylamine HCl (MEA) for 1 hr at 37° c. MEA was separated from labeled MAb in AKTAprime and the volume collected was 2800 μl. Each formulation was incubated with 4.1 mg trastuzumab in 700 μl.

### FITC Labeling of AMB8LK (Green Color)

4.1 mg AMB8LK in 1 ml were washed with sodium bicarbonate 0.165M buffer pH 9.4. 50 μl of 10 mg/ml FITC in DMF solution were added gradually to the MAb solution while stirring. The reaction was incubated for 1 hr at 4° C. To separate labeled MAb from free FITC PD10 column was used and washed with PBS-EDTA pH 7.2 (1.8 g NaHPO3 (60 mM), 4.35 g NaCl (150 mM), 0.93 g EDTA (5 Mm)). Final volume of the collected labeled MAb was 2400 μl. 15 μl of the solution were diluted 1:67 with PBS-EDTA and the sample was read in UV spectrophotometer at 280 mn (protein) and at 492 nm (FITC).

1 mg/ml IgG-1.4 Aₚᵣₒₜₑᵢₙ

0.0236 mg/ml IgG-0.033 Aₚᵣₒₜₑᵢₙ

Decree of Labeling (DOL):

Aₘₐₓ*MW/[protein]*ε_{dye}=0.003*100000/0.0236*68000=4

Labeled MAb was concentrated to 1 ml in 30K filter eppendorf (Pall), than 1.4 mg in 345 μl incubated with 6 mg 2-mercaptoethylamine HCl (MEA) for 1 hr at 37° C. MEA was separated from labeled MAb in AKTAprime and the volume collected was 320 μl. The MAb solution was concentrated to about 350 μl. The formulation was incubated with 1.4 mg AMB8LK.

### Incubation With Formulations

3 ml 30% PEG-PLA nanoparticles were incubated with 4.1 mg labeled trastuzumab and with 1.4 mg AMB8LK. Formulations were incubated under nitrogen at 4° C. for 2 nights. To separate free MAb from conjugated MAb nanoparticles were washed 3 times in 300K vivaspin.

### Formulation Characterization

### Conjugation Efficiency

The UV absorption of the formulations was read in UV spectrophotometer before and after separation. 50 ul of each nanoparticles formulation was diluted with 1 ml acetonitrile. The ratio between the results represents the conjugation efficiency (Table 2). It is noted that sulforhodamine B chloride acid labeled trastuzumab exhibits absorbance at 570 nm while FITC-labeled AMB8LK exhibits absorbance at 492 nm.

TABLE 2
Conjugation efficiency
Nanoparticles conjugated to

| Absorbance | Trastuzumab^{(a)}/ | Absorbance | Trastuzumab^{(a)}/ |
|---|---|---|---|
| at 570 nm | AMB8LK^{(b)} | at 492 nm | AMB8LK^{(b)} |
| Before | 0.019 | Before | 0.036 |
| separation | | separation | |
| After | 0.0040 | After | 0.0054 |
| separation | | separation | |
| Ratio (%) | 21 | Ratio (%) | 15 |

^{(a)}Sulforhodamine B chloride acid labeled trastuzumab
^{(b)}FITC-labeled AMB8LK

It can clearly be deduced from the results depicted in the above Table 2 that at least 21% of the initial amount of trastuzumab and 15% of the initial amount of AMB8LK antibodies are attached to the same nanoparticles. It is demonstrated that it is feasible to conjugate two different antibodies recognizing different antigens on the same nanoparticles.

### Particle Size Analysis

Mean diameter measurements was carried out utilizing an ALV Noninvasive Back Scattering High Performance Particle Sizer. Mean diameter found to be 313 ml.

### Zeta Potential Measurements

The zeta potential of the nanoparticles (in three different samples) before and after separation was measured with the Malvern zetasizer (Malvern, UK) diluted in double distilled water.

TABLE 3
Potential of the nanoparticles before and after separation

Trastuzumab conjugated nanoparticles

| Formulation | Before separation | After separation |
|---|---|---|
| Sample 1 | −24.2 | −19.8 |
| Sample 2 | −25.6 | −19.9 |
| Sample 3 | −24.2 | −20.1 |
| Mean zeta (mV) | −24.67 | −19.93 |

Fluorescence microscope showed that AMB8LK is conjugated on the surface of the nanoparticles since the particles were green colored (not shown). It should be emphasized that even if trastuzumab is attached to the same nanoparticles, it would not have been possible to visualize them because the rhodamine filter is missing.

Fluorescence microscope showed that trastuzumab is conjugated on the surface of the nanoparticles (not shown). It should be emphasized that even if AMB8LK is attached to the same nanoparticles, it would not have been possible to visualize them because the FITC filter is missing.

Thus, the same nanoparticles elicited the respective color as indicated by the filter color demonstrating the presence of both antibodies on the nanoparticles.

### (E) Characterization of Nanoparticle-Antibody Assembly

### Stability Study of the Nanoparticle-Antibody Conjugate

The stability of the coupled particles is studied in vitro by accelerated tests such as elevation of temperature, stirring and also using long term storage assessment.

The following propertiesis examined: mean diameter, distribution, zeta potential, pH and drug content using HPLC.

### In Vitro Drug Release Kinetic Evaluation

The in vitro drug release profile from the immunonanoparticles is carried out using an ultrafiltration technique at low pressure as follows: 0.4 ml of the medicated particles (containing 1-6 mg of the drug) is directly placed in a Amicon 8200 stirred vessel (Amicon, Danvers, Mass., U.S.A) containing 100 ml of release medium (maintaining sink conditions). At given time intervals, the release medium is filtered through the YM-100 ultrafiltration membrane at low pressure (less then 0.5 bar) using nitrogen gas. An aliquot of 1 ml of the clear filtrate is assayed for drug content using HPLC. Membrane adsorption and rejection must be accounted for in order to accurately measure aqueous concentrations of drug therefore validation is preformed prior to the use of the ultrafiltration technique.

### Measurement of Immunonanoparticles and Drug Uptake by the Cells

SK-BR-3 and LNCaP cells are grown to subconfluency on 24 well plates. Cells are incubated with coumarin-6 labeled nanoparticles (blank particles, DCTX loaded NPs and DCTX loaded immunoNPs) at 37° C. for different time intervals. Plates are taken for fluorescence measurements using FluoStar-Galaxy (BMG Labtechnologies) with excitation wavelength 485 nm and emission wavelength of 520 nm. Each plate is read 4 times and an average value is calculated. Wells which are not incubated with the same samples serve as a reference for total fluorescence.

For drug uptake quantification SK-BR-3 and LNCaP cells are trypsinized after reaching confluence and transferred into tubes (10⁶ cells per tube). Cells are washed and self-fluorescence are blocked with 5% BSA. Cells are incubated with coumarin-6 labeled nanoparticles (blank particles, DCTX loaded NPs and DCTX loaded immunoNPs) for different time intervals. Cells are washed, fixated and analyzed by flow cytometry.

### Pharmacokinetic Evaluation

Different particle formulations (blank particles, DCTX loaded NPs and DCTX loaded immunoNPs) made out of radiolabeled polymer [³H]-poly(lactic acid) are injected into the tail vein of healthy male BALB/c mice (20-26 g) at a volume of 5 ml/kg. At the following time intervals after injection: 5, 10, 30 min, 1, 2, 8 24, 48, 72 h, 1, 2 weeks the animals are anaesthetized with ether. Blood are then be collected from the heart and the animals are sacrificed. The heart, lung, liver, spleen, pancreas kidney, mammary glands, colon, intestine and brain are excised and rinsed with saline. Blood are centrifuged to obtain plasma. For each time interval 5 animals are used. The various organ samples are stored in plastic vials and frozen (−80° C.) until analysis. The radioactivity of the organs and plasma are measured using liquid scintillation counter for biodistribution evaluation. Docetaxel are determined either by HPLC or LC-MS.

### Pharmacological Models

The PC-3.38 human prostate cancer lines are subconfluent cultured, trypsinized and washed with PBS. Male SCID/beige mice 8 weeks of aged are anesthetized with intramuscular (i.m.) injection of ketamine 100 mg/ml and xylazine 20 mg/ml at ratio of 85:15, respectively. A lower midline abdominal incision is made, the prostate is exposed and tumor cells (5×10⁵ cells in 0.05 ml PBS) are injected into prostate as described [Honigmana A, et al. Mol Ther. 2001 September; 4(3):239-49].

The firefly luciferase gene luc, which encodes an enzyme that catalyzes the oxidation of luciferin in the presence of ATP to generate light, enable visualization of gene expression noninvasively in intact animal in the means of cooled charge-coupled device (CCCD) camera. Upon luciferin IP administration, luciferin reaches the various organs of mice and rats to generate detectable light emission [Caroline D. et al. Prostate. 59(3):292-303 (2004)]. Such bioluminescence imaging (BLI) employs noninvasive monitoring of the growth of luciferase-expressing carcinoma cells in vivo.

Mice are randomly assigned to the different treatment groups (5-10 mice per group). Different particle formulations (DCTX loaded NPs and DCTX loaded immunoNPs) are injected i.v. The marketed Taxotere® is also injected at the same dose as in the various nanoparticulate formulations to evaluate the intrinsic effect of each formulation and component. docetaxel is considered the drug of choice for prostate cancer. Tumors are measured once weekly by BLI. Histopathological examinations of the tumor injected site in case of complete tumor regression and gross examination of different organs are performed. Mice are weighed and examined for toxicity twice a week. All the data is submitted to appropriate statistical analyses. Furthermore the potential of activating human complement by the NPs formulations and by Taxotere® is evaluated using enzyme-linked immunosorbent assays (EIA) (Quidel Corporation, CA, USA).

### Example 4

### (A) NP's Preparation for In Vivo Study

The polymers PLA (MW 100,000) and mPEG-PLA (MW 100,000) (2:1) were dissolved in 50 ml acetone containing 0.2% w/v Tween 80, (Sigma, St. Louis, Mo.) at a concentration of 0.6% w/v. For loading of the drug, paclitaxel-palmitate (pcpl), 0.08% w/v of the drug was added to the polymer mixture and dissolved into the organic phase. The linker OMCCA [Octadecyl-4-(maleimidomethyl)cyclohexane-carboxylic acid] at a concentration of 0.04% w/v, was also incorporated into the organic phase. The organic phase was added to 100 ml of the aqueous phase which contains 0.25% w/v Solutol® HS 15 (BASF, Ludwigshafen, Germany). The suspension was stirred at 900 rpm over 1 h and then concentrated by evaporation to 10 ml. The formulations containing OMCCA were adjusted to pH 8.5 and incubated overnight at 4° C. under nitrogen with thiolated monoclonal antibody (MAb). All formulations were diafiltrated with 10 ml solution of 0.1% Tween 80 (Vivaspin 300,000 MWCO, Vivascience, Stonehouse, UK) and filtered through 1.2 um filter (FP 30/1.2 CA, Schleicher & Schuell, Dassel, Germany).

For the preparation of fluorescent NPs; an acetone coumarin-6 solution (Sigma, St. Louis, Mo.) at a concentration of 3×10⁻⁴% w/v was added to the organic phase before mixing with water. The formulations containing OMCCA in this particular example were incubated with the following thiolated MAbs: AMB8LK (mouse anti H-ferritin), trastuzumab (human anti HER-2) and with a combination of the two mAbs, AMB8LK and trastuzumab (molecular ratio of 1:1).

For the preparation of radiolabeled NPs 13 μCi of [³H]-pcpl were mixed with 0.02% w/v of pcpl acetone solution and added to the organic phase (prior to mixing with water resulting in a total dose of 10 mg of pcpl in the formulation described above.

### (B) Affinity of Drug Loaded Immunonanoparticles to PC-3.38 Cells

Pcpl loaded NPs conjugated to trastuzumab were prepared as described above. Human prostate cancer cell over-expressing HER 2 (PC-3.38 cells 300,000) in 2 ml medium (RPMI 1640, Biological industries, Beit Aemek, Israel) were placed on cover-slides in 12-well plates and incubated over 24 h at 37° C. and 5% CO₂ atmosphere to sub-confluency. Cells were fixated with 4% para-formaldehyde solution (Fluka, Steinheim, Switzerland) and incubated with 1% BSA solution (Sigma, St. Louis, Mo.) at ambient temperature. After the BSA solution was discarded, diluted formulations (1:100) were incubated with the cells over 2 hr at 4° C. Cells were washed 3 times with cold PBS solution (Biological industries, Beit Aemek, Israel) then, incubated with FITC labeled goat anti-human IgG (Jackson ImmunoResearch Laboratories, PA, USA). Cells were washed again with cold PBS solution, mounted on glass slides and examined with Olympus 1×70 confocal laser scanning microscope (Olympus Co. Ltd., Tokyo, Japan).

### Results

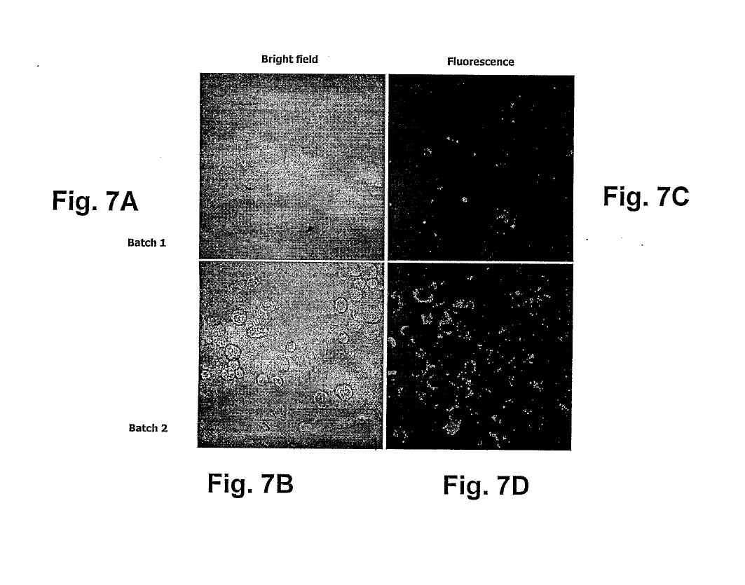

The pcpl immunoNPs conjugated to monoclonal antibody-trastuzumab exhibit affinity towards the HER 2 receptor over expressed in PC-3.38 cells as shown in FIGS. 7A-7D confirming that the conjugation process did not affect the original affinity binding of the trastuzumab.

### (C) In Vitro Uptake of Fluorescent Formulations to PC-3.38 Cells

PC-3.38 cells (300,000 cells) were grown to sub-confluency on 12-wells plates. NPs and immunoNPs were labeled with coumarin-6. Then, cells were incubated with labeled NPs and trastuzumab immunoNPs diluted 1:1000 in 1 ml culture medium at 37° C. and 5% CO₂ atmosphere over 3 h. following 3 washes with PBS cells were fixated with 4% PFA and mounted on glass slides and observed with CLSM (LSM410, Zeiss, Oberckochen, Germany).

### Results

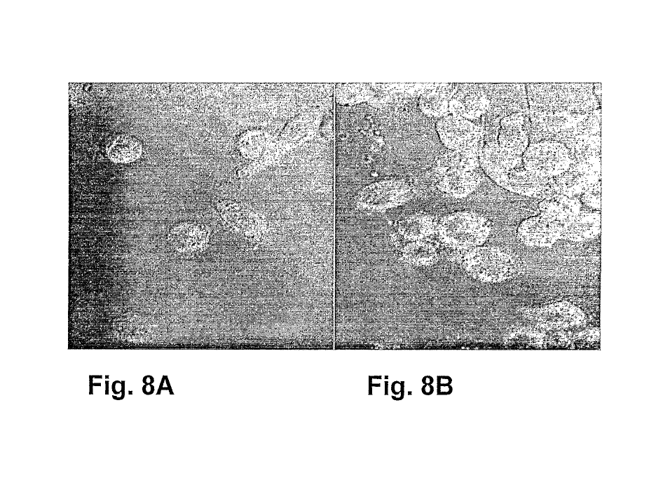

The CLSM observations show that the presence of the fluorescent immunonanoparticles in the cells cytoplasm and cells membranes are increased significantly (FIG. 8B) as compared to plain fluorescent nanoparticles (FIG. 8A).

### (D) Binding of Fluorescent Formulations to Cell Lines

Fluorescent NPs and immuno-NPs were prepared as described above. The physical properties of the formulations are presented in Table 4.

TABLE 4
Physical properties of fluorescent NPs and immunoNPs.

| | | | | Coumarin-6 |
|---|---|---|---|---|
| | | Coumarin-6 | Coumarin-6 | trastuzumab |
| | Coumarin-6 | AMB8LK | trastuzumab | and AMB8LK |
| Parameter | NPs | immunoNPs | immunoNPs | immunoNPs |
| Mean | 82 | 252 | 103 | 116 |
| diameter, | | | | |
| nm | | | | |
| MAb conc., | 0 | 580 | 670 | 730 |
| μg/ml | | | | |
| Mean zeta | −26.2 | −20.1 | −29.4 | −22.1 |
| potential, | | | | |
| mV | | | | |

### (E) Binding of Fluorescent Formulations to Cell Lines

Human prostate cancer cells (300,000, PC-3.38, over-expressing HER-2) and human pancreas cancer cells (300,000, CAPAN-1, human pancreas cancer, over-expressing H-ferritin) in 2 ml medium (RPMI 1640 and DMEM, respectively, Biological industries, Beit Aemek, Israel) were placed on cover-slides in 12-well plates and incubated over 24 h at 37° C. and 5% CO₂ atmosphere to sub-confluency. Cells were fixated with 4% para-formaldehyde (Fluka, Steinheim, Switzerland) solution and incubated with 1% BSA (Sigma, St. Louis, Mo.) solution at ambient temperature. After the BSA solution was discarded, diluted fluorescent formulations (1:2000) were incubated with the cells over 2 hr at 4° C. Cells were washed 3 times with cold PBS solution (Biological industries, Beit Aemek, Israel), mounted on glass slides and observed with Olympus 1X70 confocal laser scanning microscope (Olympus Co. Ltd., Tokyo, Japan).

### Results

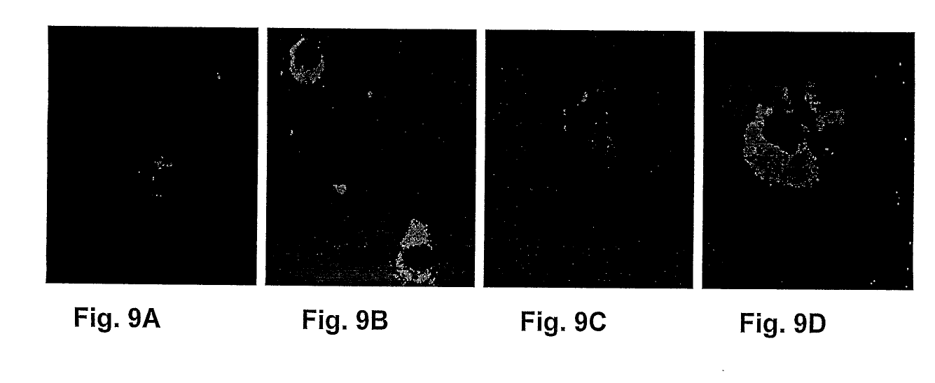

The data presented in FIGS. 9A-9D show immunoNPs conjugated to AMB8LK (FIG. 9B) or conjugated to trastuzumab (FIG. 9C) or to trastuzumab and AMB8LK in a ratio of 1:1 (FIG. 9D) that the AMB8LK immunoNPs recognized specifically the H-Ferritin antigen known to be over expressed in CAPAN-1 while the trastuzumab immunoNPs did not recognized the CAPAN-1 since they do not over-express HER-2 receptor as expected. However, when the combined immunoNPs were incubated with the CAPAN 1 cells, the NPs recognized the cells clearly demonstrating the affinity of AMB8LK was not affected by the presence and conjugation of trastuzumab to the same nanoparticles.

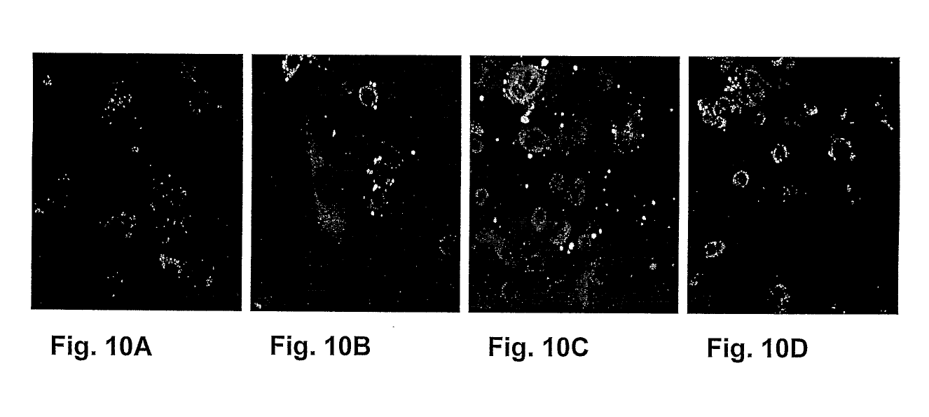

The same sets of immunoNPs were also incubated with PC3.38 known to over express the HER-2 receptor. It can clearly be deduced from the data presented in FIGS. 10A-10D that the trastuzumab conjugated NPs recognized the PC3.38 cells (FIG. 13B). Surprisingly, the AMB81k conjugated NPs also recognized the PC3.38 cells (FIG. 13C) indicating that these cells do also over-express the H-ferritin antigen.

### (F) Radiolabeled Formulations Uptake to PC-3.38 Cells

The uptake of drug from radiolabeled formulations by cells in culture was studied following incubation of the cells with preparations containing [³H]-paclitaxel-palmitate ([³H]-pcpl) at 37° C. over 3 h. PC-3.38 cells (500,000) in 2 ml medium (RPMI 1640) were placed in 12-well plates and incubated for 24 h at 37° C. and 5% CO₂ atmosphere. In each well, the total initial radioactivity used was 45 μCi of [³H]-pcpl solution, [³H]-pcpl loaded NPs and [³H]-pcpl loaded NPs conjugated to trastuzumab, equivalent to 22 μg of pcpl. Following incubation over 3 hr at 37° C. and 5% CO₂ atmosphere, the formulations were discarded and the cells were washed 3 times with PBS. Cells were trypsinized and treated with sodium hydroxide solution. The radioactivity was monitored in Ultima-Gold scintillation mixture (Packard Instruments, Boston, Mass., USA) in a beta counter (Kontron Instruments, Milan, Italy).

### Results

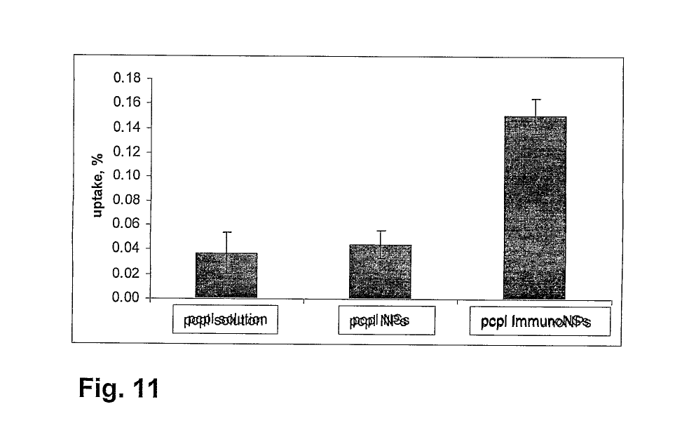

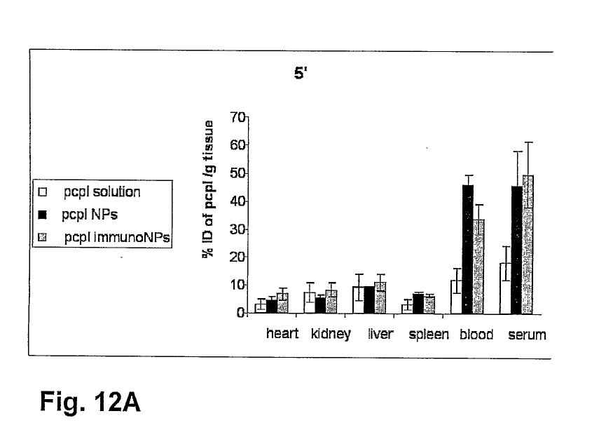

The percentage of the uptake was calculated from the total radioactivity as presented in FIG. 11. The uptake percentage of pcpl immunoNPs was markedly higher from the uptake percentage of pcpl NPs and pcpl solution. These findings establish the specific targeting of drug loaded colloidal carrier to desired tissue by the means of MAbs.

### (G) Pharmacokinetics and Biodistribution of ImmunoNPs in Mice.

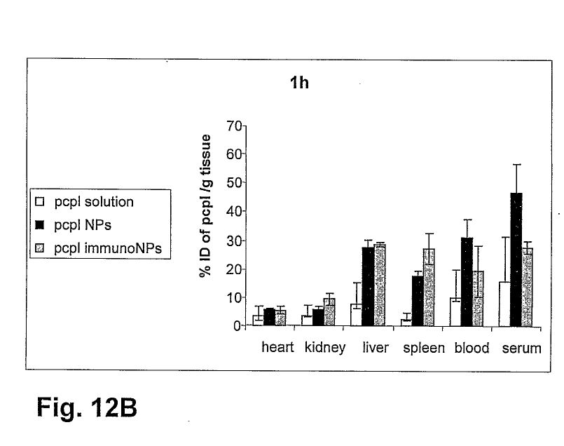

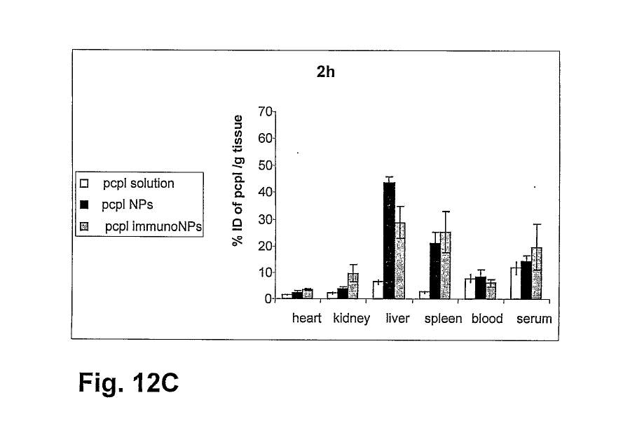

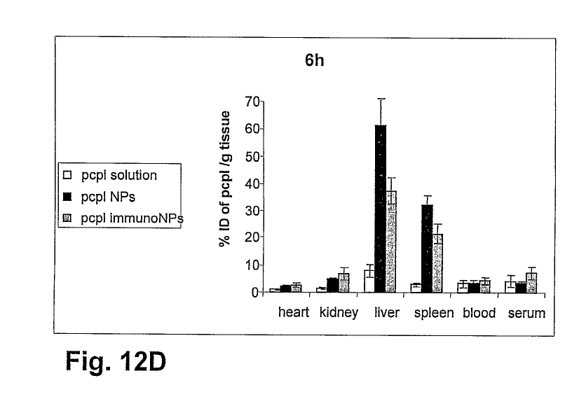

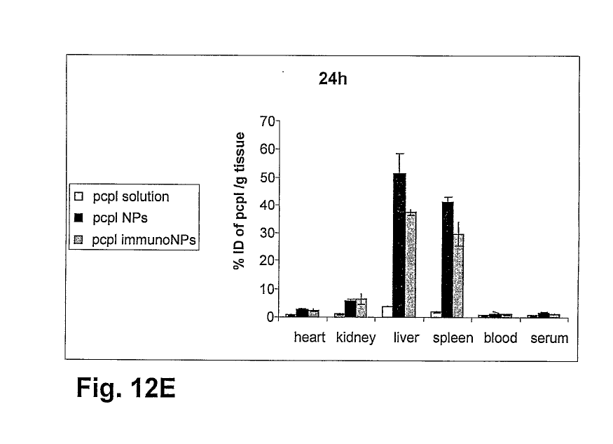

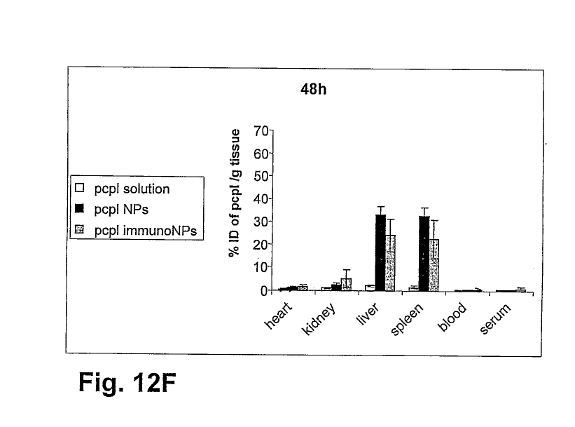

The biodistribution and pharmacokinetic profile of [³H]-pcpl in cremophor EL:ethanol solution, [³H]-pcpl loaded NPs and [³H]-pcpl loaded NPs conjugated to trastuzumab were studied in male Balb/C mice 8 weeks of age. Four mice were assigned to each group in which a radioactive dose of 0.225 μCi of [³H]-pcpl equivalent to a total dose of 7.5 mg/kg of pcpl were injected into the tail vein in one bolus dose. Animals were sacrificed by cervical dislocation and tissues of interest (i.e. heart, liver, spleen, kidneys, blood and plasma) were identified and removed using simple surgery techniques. Following washing with 1 ml sterile saline (0.9% sodium chloride), tissues were weighed, incubated with 1 ml Solvable tissue solubilizer (Packard, Groningen, The Netherlands), tissues and discolored with 30% hydrogen peroxide solution (Fluka, Steinheim, Switzerland). The radioactivity was monitored in Ultima-Gold scintillation mixture (Packard, Groningen, The Netherlands) in a beta counter (Kontron Instroments, Milan, Italy). Concentrations of [³H]-pcpl in blood were plotted against time on log-linear graph (FIG. 6) while the pharmacokinetic parameters of the drug were further studied by noncompartmental analysis using the WinNonlin® Professional software version 4.0.1 and are presented in Table 5. The biodistribution of [³H]-pcpl in tissues of interest is presented at selected time intervals as the percent fraction of drug in tissue from the drug initial dose normalized to gram tissue (FIGS. 12A-12F).

### Results

The data presented in FIGS. 12A-12F and Table 5 shows that the residence time of the pcpl NPs and immunoNPs is much more extended in blood than pcpl solution. Both nanoparticulate delivery systems succeeded in prolonging drug release in the circulation and masked the intrinsic pharmacokinetic profile of pcpl owing to the stealth character of the nanoparticles. In fact the steric hindrance elicited by the PEG moieties located on the NP surfaces prevent the opsonization of the NPs and allows prolonged circulation time. It is interesting to note that the terminal half life of the pcpl NPs and immunoNPs was 14.6 and 20 h respectively; significantly higher than the half life of 8.3 h elicited by the pcpl solution. In addition, the immunoNPs exhibited a higher half life value than the NPs probably as a result of the conjugation of trastuzumab on the NP surfaces. The antibody which is a macromolecule probably confers some additional steric hindrance and increase the residence time compared to the normally PEGylated NPs as noted from the data presented in Table 5. Both pcpl NPs and immunoNPs increased markedly the Cₘₐₓ and AUC values as compared to the AUC value at infinity of pcpl solution (Table 5). However, there was no difference in the Cₘₐₓ and AUC values between pcpl NPs and pcpl immunoNPs.

TABLE 5
Pharmacokinetics parameters of [³H]-pcpl formulations

| | Terminal | | | Mean |
|---|---|---|---|---|
| | half | AUC | Cₘₐₓ, | Residence |
| Parameter | life (hr) | (h*μg/ml) | μg/ml | Time (hr) |
| pcpl solution | 8.3 | 84.5 | 9.7 | 9.1 |
| pcpl NPs | 14.6 | 132.3 | 51.1 | 12.2 |
| pcpl immunoNPs | 20 | 137.5 | 45.2 | 15.3 |

FIGS. 12A-12F show the organ distribution of the three preparations over different time points up to 48 hours in healthy animals. It can clearly be deduced that the pcpl NPs and ImmunoNPs are eliminated by the reticulo endothelial system mainly the liver and spleen since more than 50% of the initial dose are located in both the liver and spleen at 48 h post injection. No preferential NPs uptake by the erythrocytes is observed since there was no difference in the profile of the NPs between blood and serum.

Tumor bearing mice over-express the HER2 receptor and therefore, conducting the same assay as above, however with SCID/beige mice (i.e. tumor-bearing mice) will show that radio-labeled targeted NP's of the invention will accumulate at the tumor area.

While this invention has been shown and described with reference to preferred embodiments thereof, it will be understood by those skilled in the art that many alternatives, modifications and variations may be made thereto without departing from the spirit and scope of the invention. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference.

1.-31. (canceled)

32. A delivery system comprising:
(i) a polymer-based nanoparticle; and
(ii) a linker comprising a first portion non-covalently anchored to said nanoparticle, wherein at least part of said first portion comprises a lipophilic segment embedded in said nanoparticle; and a second portion comprising a maleimide compound exposed at the outer surface of said nanoparticle.

33. The delivery system of claim 32, wherein said linker is an amphipathic molecule.

34. The delivery system of claim 32, wherein said lipophilic portion comprises a hydrocarbon or a lipid comprising at least 8 carbons.

35. The delivery system of claim 32, wherein said linker has the following general formula (I): 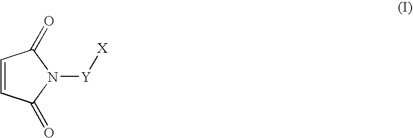
wherein
Y represents a heteroatom, a C₁-C₂₀ alkylene or alkenylene, a C₅-C₂₀ cycloalkylene or cycloalkenylene, C₆-C₂₀ alkylene-cycloalkykylene, wherein one of the carbon atoms in said alkylene or alkenylene may be replaced by a heteroatom;
X represents a carbonyl containing moiety selected from —C(O)—R₁, —C(O)—NH—R₁, —C(O)—O—C(O)—R₁, C(O)NH—R₂—R₁, or —C(O)—NH—R₂—C(O)—NH—R₁, wherein R₁ represents a hydrocarbon or a lipid comprising at least 8 carbons and R₂ represents a hydrophilic polymer.

36. The delivery system of claim 35, wherein said R₁ is a lipid selected from mono or diacylglycerol, a phospholipid, a sphingolipid, a sphingophospholipid or a fatty acid.

37. The delivery system of claim 35, wherein said Y is an alkylene cyclohexane.

38. The delivery system of claim 37, wherein said Y represents an alkylene cycloalkykylene having the formula —CH₂—C₆H₁₀—; X represents a carbonyl containing moiety having the formula —C(O)—NH—R₁, wherein R₁ is a fatty acid.

39. The delivery system of claim 35, wherein said linker is selected from Octadecyl-4-(maleimidomethyl)cyclohexane-carboxylic amide (OMCCA); N-1 stearyl-maleimide (SM); succinimidyl oleate; 1,2-Distearoyl-sn-Glycero-3-Phosphoethanolamine-N-[Maleimide(Polyethylene Glycol)2000]; and mixtures thereof.

40. The delivery system of claim 32, wherein said linker is octadecyl-4-(maleimideomethyl)cyclohexane-carboxylic amide (OMCCA).

41. The delivery system of claim 32, comprising an active agent embedded, impregnated or encapsulated in said particle, or adsorbed to the surface of the particle.

42. The delivery system of claim 32, wherein said polymer is a biodegradable polyester selected from polyhydroxybutyric acid, polyhydroxyvaleric acid, polycaprolactone, polyesteramide, polycyanoacrylate, poly(amino acids), polycarbonate, polyanhydride, poly alkylcyanoacrylate and mixtures of same.

43. The delivery system of claim 42, wherein said polyester is polylactide (PLA), polyglycolide, polylactide-polyglycolide, poly(lactide-co-glycolide) or polyethylene glycol-co-lactide (PEG-PLA).

44. The delivery system of claim 32, wherein said hydrophilic polymer is selected from polyethylene glycol (PEG), polysialic acid, polylactic (also termed polylactide), polyglycolic acid (also termed polyglycolide), apolylactic-polyglycolic acid, polyvinyl alcohol, polyvinylpyrrolidone, polymethoxazoline, polyethyloxazoline, polyhydroxyethyloxazoline, polyhydroxypropyloxazoline, polyaspartamide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose.

45. The delivery system of claim 44, wherein said hydrophilic polymer is PEG having an average molecular weight in the range between 2,000 and 5,000 Da.

46. The delivery system of claim 32, comprising one or more targeting agents, each covalently bound to said maleimide compound.

47. The delivery system of claim 46, wherein said targeting agent is a polymer selected from an amino acid-based, nucleic acid-based, or saccharide based polymer and combination of same.

48. The delivery system of claim 47, wherein said targeting polymer is selected from ligands, antibodies, antigens, glycoproteins.

49. The delivery system of claim 46, wherein said targeting agent is a low molecular weight ligand.

50. The delivery system of claim 48, comprising at least two antibodies or antibody fragments, each with different binding specificity.

51. The delivery system of claim 48, wherein said antibody is a genetically engineered antibody selected from trastuzumab, AMB8LK or a combination of same.

52. A composition comprising the delivery system of claim 32 in combination with a pharmaceutically acceptable carrier.

53. A method for treating or preventing a disease or disorder, the method comprises providing a subject in need, an amount of the delivery system claim 32, wherein said delivery system comprises a drug, the amount of the drug being effective to treat or prevent said disease or disorder.

54. A method of imaging in a subject's body a target cell or target tissue, the method comprising:
(a) providing said subject with a delivery system of claim 32 carrying a contrasting agent, wherein the nanoparticles are associated with one or more targeting agents effective to target said delivery system to said target cell or target tissue;
(b) imaging said contrasting agent in said body.

55. The method of claim 54, wherein said contrasting agent is coumarin-6.

56. The method of claim 53, wherein said delivery system comprises a anti-cancer drug embedded in said particle.
